(19) United States
(12) Patent Application Publication
(10) Pub. No.: US 2018/0030519 A1
(43) Pub. Date: Feb. 1, 2018

(54) METHODS AND SYSTEMS FOR DETECTING TARGET NUCLEIC ACIDS

(71) Applicants: Vladimir I. Bashkirov, Davis, CA (US); Theofilos Kotseroglou, Hillsborough, CA (US)

(72) Inventors: Vladimir I. Bashkirov, Davis, CA (US); Theofilos Kotseroglou, Hillsborough, CA (US)

(21) Appl. No.: 15/437,006

(22) Filed: Feb. 20, 2017

Related U.S. Application Data
(60) Provisional application No. 62/297,826, filed on Feb. 20, 2016.
(60) Provisional application No. 62/300,623, filed on Feb. 26, 2016.

(51) Int. Cl. C12Q 1/68; C07H 21/04

(52) U.S. Cl. CPC C12Q 1/6811; C12Q 1/6816; C12Q 1/6883; C07H 21/04; C12Q 1/6834; C12Q 1/6832; C12Q 2600/112; C12Q 2600/118; C12Q 2600/158

(57) ABSTRACT

The present invention provides methods and systems for nucleic acid detection and identification.

### CROSS REFERENCE TO RELATED APPLICATIONS

This application claims the benefit of priority under 35 U.S.C. 119(e) to U.S. Application No. 62/297,826 filed Feb. 20, 2016 and U.S. Application No. 62/300,623 filed Feb. 26, 2016.

### TECHNICAL FIELD

This disclosure generally relates to methods and systems for detecting nucleic acids.

### BACKGROUND

The detection of small amounts of target nucleic acid can be performed by various qualitative and/or quantitative methods, most of which use the two major strategies—signal amplification, or target nucleic acid amplification. Among the latter methods, the qPCR method, which employs various designs of fluorescently labeled probes for increased specificity, is considered to be the gold standard for various medical and non-medical applications. However, there is a need for methods and composition to detect nucleic acids in a point-of-use or point-of-care setting without requiring bulky, expensive, and/or delicate equipment.

### SUMMARY

In one aspect, a method of amplification-free target nucleic acid sequence detection is provided. Such a method typically includes (a) providing a sample comprising at least one target nucleic acid sequence; (b) contacting the sample with a probe comprising a nucleic acid moiety and a positively-charged tag, wherein the nucleic acid moiety is complementary to at least a portion of the target nucleic acid sequence, wherein the contacting is performed under conditions in which a probe-target complex is formed between the nucleic acid moiety and the complementary target nucleic acid sequence; (c) cleaving the probe within the probe-target complex to release the detectable positively-charged tag; and (d) detecting the movement of the released positively-charged tag through a nanopore based on a change in an electrical signal; wherein a change in the electrical signal indicates the presence of the target nucleic acid sequence in the sample.

In some embodiments, the probe further comprises a scissile linkage selected from the group consisting of RNA sequences, DNA sequences, and abasic nucleotide sequences. A scissile linkage can include at least one RNA residue. A scissile linkage can include an oxidized purine or an oxidized pyrimidine. A scissile linkage can include an apurinic site or an apyrimidinic site. A scissile linkage can include deoxyuridine, 5-hyroxyuracil, 5-hydroxymethyluracil, or 5-formyluracil. A scissile linkage can be cleaved by type 1 ribonuclease H or type 2 ribonuclease H. A scissile linkage can be cleaved by a combination of DNA N-glycosylase and DNA AP-lyase activity. A scissile linkage can be cleaved by DNA AP-lyase activity or an endodeoxyribonuclease. A scissile linkage can be cleaved by a combination of DNA N-glycosylase and endodeoxyribonuclease, or a combination of DNA N-glycosylase and DNA AP-lyase activity.

In some embodiments, the method can further include: in step (b), further contacting the sample with a primer, wherein the primer is complementary to at least a portion of the target nucleic acid sequence that is upstream of the portion of the target nucleic acid sequence to which the probe is complementary, wherein the contacting is performed under conditions in which a primer-target complex is formed between the primer and the complementary target nucleic acid sequence; and in step (c), contacting the sample comprising the primer-target complex with DNA polymerase under conditions in which primer extension occurs.

In some embodiments, the probe is cleaved during primer extension by the DNA polymerase. In some embodiments, the cleavage is mediated by the 5′ nuclease activity of the DNA polymerase.

In some embodiments, the extension by the DNA polymerase is limited by the number of dNTP types present in the reaction. In some embodiments, the number of dNTPs present in the reaction is one, or two, or three of the four dNTPs.

Such a method can further include: in step (b), further contacting the sample with at least two primers, wherein the at least two primers are complementary to portions of the target nucleic acid sequence that flank the portion of the target nucleic acid sequence to which the probe is complementary, wherein the contacting is performed under conditions in which primer-target complexes are formed between the at least two primers and the complementary target nucleic acid sequences; and in step (c), amplifying the target nucleic acid sequence between the at least two primers using a DNA polymerase.

In some embodiments, amplifying the target nucleic acid sequence comprises a polymerase chain reaction (PCR) or an isothermal reaction. In some embodiments, the probe is cleaved by the DNA polymerase during amplification. In some embodiments, the cleavage is mediated by the 5′ flap endonuclease activity of the DNA polymerase. In some embodiments, cleavage results in detection of the detectable positively-charged tag, which is indicative of the replication of target amplicon.

In some embodiments, the probe comprises a net negative charge. In some embodiments, the detectable positively-charged tag comprises a net positive charge before and after being released. In some embodiments, the detectable positively-charged tag comprises a positively charged nucleic acid moiety, a non-nucleic acid moiety, or a combination thereof before and after being released.

In some embodiments, the contacting step comprises contacting the sample with a plurality of probes that each are complementary to at least two different target nucleic acid sequences and that each have a different positive charge (amount or type), and wherein the electrical signal (type/amount) is able to distinguish the at least two different target nucleic acid sequences. In some embodiments, the cleaving step comprises cleaving the probe enzymatically.

In some embodiments, the detectable positively-charged tag passes through the nanopore. In some embodiments, the detectable positively-charged tag is detectable by its charge, shape, size, or any combination thereof. In some embodiments, the detecting step further comprises identifying the detectable positively-charged tag. In some embodiments, the method further comprises correlating the identified tag with the presence of the corresponding target nucleic acid sequence. In some embodiments, the method further comprises correlating the amount/level of electrical signal with the amount of the target nucleic acid sequence in the sample.

In some embodiment, the detectable positively-charged tag is detected using an ion-sensitive field-effect transistor. In some embodiments, the method uses a computer processor.

In another aspect, a method of detecting a target nucleic acid in a sample using a target-specific probe is provided. Such a method typically includes (a) providing a sample comprising a plurality of single-stranded nucleic acid fragments; (b) circularizing, intra-molecularly, the single-stranded nucleic acids to produce single-stranded circles; (c) contacting the single-stranded circles with at least one probe-specific oligonucleotide primer under hybridization conditions in which the at least one probe-specific oligonucleotide primer hybridizes to the complementary sequence in the single-stranded circles and forms double-stranded primer-circle complexes; (d) contacting the double-stranded primer-circle complexes with an enzyme under conditions in which rolling circle replication occurs; (e) contacting the products of the rolling circle replication with a target-specific dye-labeled detector-probe under conditions in which the target-specific dye-labeled detector-probe hybridizes to the complementary sequence in the products of the rolling circle replication; and (f) detecting the target-specific dye-labeled detector-probe, wherein the presence of the target-specific dye-labeled detector-probe indicates the presence of the target nucleic acid in the sample.

In some embodiments, the target specific probe is bound to a solid support. In some embodiments, the circularization step is mediated by a single-stranded DNA ligase. In some embodiments, the method further comprises enzymatically digesting uncircularized linear nucleic acids to enrich for single-stranded circles. In some embodiments, the method further comprises depositing the products of the rolling circle replication on a solid support.

In some embodiments, the detecting step is performed using imaging. In some embodiments, the detecting step comprises depositing the product of rolling circle replication on the surface of a solid support.

In some embodiments, the method further comprises quantitating the target-specific dye-labeled detector probe and correlating the amount of target-specific dye-labeled detector probe with the amount of the target nucleic acid in the sample.

In some embodiments, the method is used for prenatal testing for detection of fetal aneuploidies and further comprises: wherein the plurality of single-stranded nucleic acid fragments in the sample comprises fetal and maternal cell-free genomic DNA; wherein the at least one target-specific probe comprises a plurality of chromosome-specific probes, wherein the plurality of chromosome-specific probes comprises a first set of probes comprising at least 100 different nucleic acid sequences corresponding to a first chromosome being tested for aneuploidy, and a second set of probes comprising at least 100 different nucleic acid sequences corresponding to a reference chromosome, wherein the first chromosome being tested for aneuploidy and the reference chromosome are different; wherein the at least one target-specific probe comprises a plurality of chromosome-specific probes; wherein the at least one probe-specific oligonucleotide primer comprises a plurality of chromosome-specific oligonucleotide primers, wherein the plurality of chromosome-specific oligonucleotide primers comprises at least one chromosome-specific oligonucleotide primer specific for single-stranded circles derived from the first chromosome being tested for aneuploidy, and at least one chromosome-specific oligonucleotide primer specific for single-stranded circles derived from the reference chromosome; amplifying, selectively, the double-stranded primer-circle complexes to generate linear single-stranded products, wherein the target-specific dye-labeled detector-probe is a plurality of chromosome-specific dye-labeled detector-probes, wherein the plurality of chromosome-specific detector-probes comprises at least one chromosome-specific detector-probe that is complementary to a chromosome-specific probe from the first chromosome being tested for aneuploidy, and at least one chromosome-specific detector-probe that is complementary to a chromosome-specific probe from the reference chromosome, wherein the plurality of chromosome-specific dye-labeled detector-probes specific for the first chromosome being tested for aneuploidy is labeled with a first fluorescent dye and the plurality of chromosome-specific dye-labeled detector-probes specific for the reference chromosome is labeled with a second fluorescent dye, wherein the presence of the chromosome-specific dye-labeled detector-probe comprising the first fluorescent dye indicates the presence of fetal aneuploidy.

In some embodiments, the plurality of chromosome-specific probes shares a common custom sequence. In some embodiments, the common custom sequence comprises a region that is complementary to the chromosome-specific oligonucleotide primer and a region that is complementary to the chromosome-specific dye-labeled detector-probe.

Representative fetal aneuploidies include, without limitation, trisomy 21, trisomy 18, trisomy 13, monosomy X, triple X syndrome, XYY syndrome, and XXY syndrome.

In another aspect, a composition is provided that includes at least one set of chromosome-specific oligonucleotide primers complementary to at least two different human chromosomes, comprising: a first set of chromosome-specific oligonucleotide primers complementary to a plurality of target sequences from a first chromosome, and a second set of chromosome-specific oligonucleotide primers complementary to a plurality of target sequences from a second chromosome.

In another aspect, a composition is provided that includes at least one set of chromosome-specific dye-labeled detector-probes for detecting at least two human chromosomes, comprising: a first set of chromosome-specific dye-labeled detector-probes complementary to a plurality of probe-specific oligonucleotide primers specific to a first chromosome, and a second set of chromosome-specific dye-labeled detector-probes complementary to a plurality of probe-specific oligonucleotide primers specific to a second chromosome.

In yet another aspect, a kit is provided that includes both of the compositions described herein.

Provided herein are the methods and systems of electronic detection of target nucleic acid comprising nucleic acid “amplification-free” and “with-amplification” methods and detection composition, including a probe with electronically detectable positively-charged tag; and a system of detection of the positively charged tag comprising a microfluidic device with the integrated nanopore detector comprising temperature control, two chambers, a circuit board with amplifier, an electrically resistive barrier with at least one nanopore, and signal processing software; kits for performing target amplification and nanopore sensor detection.

In one aspect, methods of detection of target nucleic acid are provided. In some embodiments, the method is the method of direct target detection which comprises: providing a sample comprising at least one polynucleotide sequence, providing a probe comprising a moiety complementary to the target sequence and a detectable positively-charged tag, providing conditions for hybridization of probe to target polynucleotide sequence to form a probe-target complex, and for subsequent hybridization-dependent enzymatic cleavage of detectable tag, wherein tag is released from a probe, providing conditions for said released tag flow through the nanopore, detecting the tag with the aid of electrode, wherein the tag is detected by generation of electrical signal subsequent to being released from said probe; and wherein said detecting of tag further comprises identifying said tag; and wherein detection of specific detectable tag in turns detects the cleavage event and therefore the target, correlating the detected electrical signal with an amount of the target polynucleotide sequence present in the sample.

In some embodiments, the target polynucleotide sequence of step (a) resides on double-stranded or single-stranded polynucleotide fragments. In some embodiments, the target polynucleotide fragments of step (a) are deoxyribonucleotide or ribonucleotide acids. In some embodiments, the target polynucleotide fragments comprise various species of viral, bacterial, fungal, and higher eukaryote DNA and RNA.

In some embodiments, the probe of step (b) comprises negatively charged oligonucleotide sequence complementary to the target and positively-charged tag. In some embodiments, the positively-charged tag is attached to the 5′-, or 3′-end of negatively charged complementary moiety. In some embodiments, two different tags are attached to the complementary moiety of the probe: one at the 5′ end and another to the 3′-end. In some embodiments, for multiplexed target detection the tags are different in the number of positive charges and/or in the chemical structure to provide the means for the differential tag identification. In some embodiments, differences in chemical structure further comprise the difference in mass and shape of the chemical compound. The positively-charged tag can be cleaved off upon probe hybridization to the target sequence.

In some embodiments, the chemical and thermal conditions are provided for hybridization of the probe of step (b) to the target polynucleotide sequence to form a probe-target complex of step (c). In some embodiments, the conditions for hybridization-dependent cleavage of detectable tag are provided, wherein tag is released from a probe. In some embodiments, the cleavage of the tag is dependent on both the hybridization of the probe to the target polynucleotide and the enzyme-catalyzed breakage of scissile linkage present in the probe. In some embodiments, the cleavage is within the moiety of the probe complementary to the target, and is adjacent to the junction between this moiety and detectable tag. In some embodiments, the released tag contains several nucleotides (negatively charged) derived from the target complementary moiety of the probe, but the overall global charge of the released detectable tag is positive.

In some embodiments, at step (d) the electrical potential is applied across electrolyte solution-containing cis- and trans-chambers of the microfluidic chip, wherein the chambers are separated by electrically resistive barrier with at least one embedded nanopore. This provides the conditions for the net positively charged tag to move from cis-chamber (+) to trans-chamber (−) by flow through the nanopore, while the uncleaved probe with the net negative charge, negatively charged primers, target nucleic acid, and amplification products remain in cis-chamber. In some embodiments, the electric potential is applied continuously for the whole duration of the cleavage reaction, or in pulses at certain time intervals (real-time detection mode). In some embodiments, the conditions for released tag flow through the nanopore provided after the completion of cleavage reaction (end-point detection mode).

In some embodiments, the released tag blocks the ionic current through the nanopore in different extent and duration, thus, producing the change in the conductance. In some embodiments, the electronic change is different for each tag, thereby identifying said tag. In some embodiments, the detection of the tag in turns detects the cleavage event and therefore the presence of the target nucleic acid.

In some embodiments, the number of electronic changes corresponds to the number of tags flowing through the nanopore, thereby correlating the number of detected electrical signals with an amount of the target polynucleotide sequence present in the sample. In some embodiments, the extent of conductance changes, being a tag identifier, correlates with the amount of particular target sequence in the sample, thereby allowing quantification of each target in the sample.

In another aspect, detection of multiple targets simultaneously (called multiplexing) are provided. In some embodiments, the detection of the targets is performed as described herein.

In another aspect, additional methods of detection of target nucleic acid are provided. In some embodiments, the method is the method of target detection using nucleic acid amplification, and it comprises: providing a sample comprising at least one polynucleotide sequence, providing at least one pair of nucleic acid amplification primers, providing a probe comprising a moiety complementary to the target sequence and a detectable positively-charged tag, performing a polymerase driven nucleic acid amplification, providing conditions for hybridization of probe to target polynucleotide sequence to form a probe-target complex, and subsequent hybridization-dependent enzymatic cleavage of detectable tag, wherein tag is released from a probe, providing conditions for said released tag flow through the nanopore; detecting the tag with the aid of electrode, wherein the tag is detected by generation of electrical signal subsequent to being released from said probe; and wherein said detecting of tag further comprises identifying said tag; and wherein detection of specific detectable tag in turns detects the cleavage event and therefore the replication of target amplicon, correlating the detected electrical signal with an amount of the target polynucleotide sequence present in the sample.

In some embodiments, the target nucleic acid is amplified using at least a pair of oligonucleotide primers of step (b) to select the location and the size/length of exponentially amplified DNA region, amplicon. In some embodiments, one primer can be used to linearly amplify the target nucleic acid.

In some embodiments, the polymerase driven nucleic acid amplification of step (d) is exponential, e.g. polymerase chain reaction (PCR). In some embodiments, the polymerase driven nucleic acid amplification of step (d) is linear, e.g. polymerase thermo cycling reaction with one primer. In some embodiments, the polymerase driven nucleic acid amplification of step (d) is the thermocycling PCR. In some embodiments, the polymerase driven nucleic acid amplification of step (d) is the isothermal amplification reaction, e.g. Recombinase Polymerase Amplification (RPA).

In some embodiments, the chemical and thermal conditions are provided for hybridization of the probe of step (c) to the target polynucleotide sequence to form a probe-target complex of step (e). In some embodiments, the conditions for hybridization-dependent cleavage of detectable tag are provided, wherein tag is released from a probe. In some embodiments, the cleavage of the tag is dependent on both the hybridization of the probe and polymerase-catalyzed cleavage of the probe during DNA synthesis, wherein the position of the cleavage is not precisely fixed. In some embodiments, the cleavage is performed by enzymes other than DNA polymerase and its position within complementary part of the probe is fixed and determined by the position of scissile linkage of the probe. In some embodiments, the cleavage of step (e) is within the moiety of the probe complementary to the target and is adjacent to the junction between this moiety and detectable tag. In some embodiments, the released tag contains several nucleotides (negatively charged) derived from the target complementary moiety of the probe, but the overall global charge of the released detectable tag is positive.

In some embodiments, the released tag blocks the ionic current through the nanopore in different extent, thus, producing the change in the conductance. In some embodiments, the electronic change is different for each tag, thereby identifying said tag. In some embodiments, the detection of the tag in turns detects the cleavage event and therefore the replication of target amplicon.

In yet another aspect, a system of target nucleic acid detection comprising a microfluidic device with the input port and integrated nanopore detector (sensor) chip. The device comprises the temperature control, two (cis- and trans-) chambers with the corresponding electrodes in contact with conductive solution; an electrically resistive barrier with the embedded nanopore with the diameter on nanometer scale separating two electrolyte solutions, the integrated sensing circuit, and signal processing software. In some embodiments, the nanopore can be a solid-state nanopore, in other embodiments it can be a biological nanopore. In some embodiments multiple nanopore detectors may form a nanopore array. In some embodiments, the nanopore detectors are individually addressable.

In yet another aspect, a conductance measurement system is presented comprising: (a) an electrically resistive barrier separating two chambers with electrolyte solutions; (b) said electrically resistive barrier comprises at least one nanopore; (c) at least one probe with a tag in electrolyte solution in cis-chamber; (d) said at least one nanopore being enabled to allow an ionic current to be driven across electrolyte solutions by an applied potential; (e) said at least one target nucleic acid and biochemical reaction components configured to perform amplification-free direct detection, or DNA amplification coupled detection, and to release detectable tag from the probe; (f) a means of measuring the ionic current; (g) a means of recording the conductance time course as a time series.

In yet another aspect, a method is provided to delineate segments of a conductance time series into regions statistically consistent with the conductance of unobstructed pore and a pore transiently obstructed by a flowing tag(s), and to quantify tags by type, and translate these data into amount of target nucleic acid present in the sample.

In yet another aspect, kits for performing the methods of detection of nucleic acids as described herein are provided.

Also provided herein are the methods and compositions of detection of target nucleic acid in the sample comprising fragments of nucleic acid. Some methods comprise the circularization of the target-specific probe and subsequent probe amplification to detect and enumerate the target. Other methods comprise the circularization of the fragments of nucleic acid present in sample, selective linear amplification of circularized fragments comprising the target nucleic acid, and the detection of amplification product. Also methods of Non-Invasive Prenatal Testing (NIPT) using cell-free DNA circulating in maternal blood to detect common chromosomal aneuploidies are provided. The compositions may comprise multiple target-specific probe sets, probe set-specific detector-probes, and target specific detector probes.

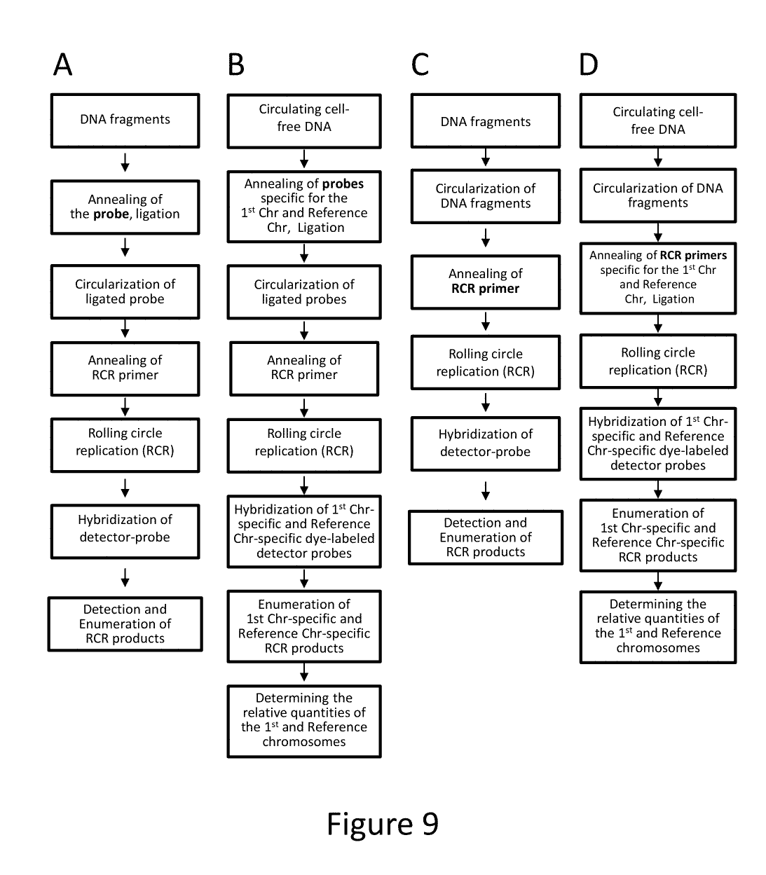

In one aspect, method of detection of target nucleic acid in the sample using target-specific probe is provided (FIG. 9A), said method comprising: providing a sample comprising plurality of polynucleotide fragments; providing denaturing conditions under which the polynucleotide fragments are converted to single-stranded form; contacting the sample with at least one target-specific probe; providing conditions for annealing/hybridization and ligation, under which said conditions the target-specific probe hybridizes to its target sequence within nucleic acid fragment and generates the ligation product, each ligation product being a linear polynucleotide comprising at least one ligation junction; providing conditions for intramolecular circularization of linear polynucleotide, comprising conditions for ligation of the free ends of said linear polynucleotide, to result in formation of single-stranded circle; enriching single-stranded circles by enzymatically digesting un-circularized linear nucleic acids; contacting the single-stranded circles with the probe-specific, or target-specific oligonucleotide primer, comprising annealing conditions under which the primer hybridizes to the complementary sequence of the circle to form a double stranded primer-circle complex suitable for initiation of DNA synthesis; providing conditions for the rolling circle replication of the primer-circle complex; detecting the product of rolling circle replication, comprising depositing of said product on solid phase, hybridizing of said product with target-specific dye-labeled detector-probe, and imaging, wherein detecting of said product indicates the presence of the target polynucleotide in the sample; and enumerating the products with detected dye-specific signal, and correlating the detected products of rolling circle replication with an amount of the target polynucleotide sequence present in the sample.

In some embodiments, a sample of (a) comprises fragmented deoxyribonucleic or ribonucleic acids. In some embodiments, the nucleic acid fragments comprise various species of viral, bacterial, yeast, fungal, higher eukaryote, or human DNA and RNA.

In some embodiments, the fragments of ribonucleic acids are converted to the deoxyribonucleic acid by the process of reverse transcription, wherein the ribonucleic acid template strand is digested by ribonuclease.

In some embodiments, conditions for dephosphorylation of the ends of polynucleotide fragments comprising treatment with phosphatase are provided prior to step (c).

In some embodiments, the target-specific probe of step (c) comprises the left arm oligonucleotide containing 5′ end moiety non-complementary to the target sequence and 3′ end moiety complementary to the target sequence, and the right arm oligonucleotide containing 5′ end moiety complementary to the target sequence and 3′ end moiety not complementary to the target sequence, so that upon hybridization of the left arm and the right arm with the target fragment the double-stranded complex forms, wherein 3′end of the left arm is positioned in juxtaposition to the 5′ end of the right arm wherein under the conditions of ligation the 3′ end of the left arm is ligated to the 5′ end of the right arm to form a ligation junction, thus, generating the product of ligation comprising continuous linear strand of nucleic acid.

In some embodiments, the target-specific probe of step (c) comprises the left arm oligonucleotide containing 5′ end moiety non-complementary to the target sequence and 3′ end moiety complementary to the target sequence, and the right arm oligonucleotide containing 5′ end moiety complementary to the target sequence and 3′ end moiety not complementary to the target sequence, and the bridge oligonucleotide complementary to the target sequence, so that upon hybridization of the left arm, the right arm, and the bridge with the target fragment the double stranded complex forms, wherein 3′end of the left arm is positioned in juxtaposition to the 5′end of the bridge oligonucleotide, and 5′ end of the right arm is positioned in juxtaposition to the 3′ end of the bridge oligonucleotide, wherein under the conditions of ligation the 3′ end of the left arm is ligated to the 5′end of the bridge to form the first ligation junction, and the 3′ end of the bridge is ligated to the 5′ end to the right arm to form the second ligation junction, thus, generating the product of double ligation comprising continuous linear strand of nucleic acid.

In some embodiments, the probe's left arm and right arm moieties, which are not complementary to the target, each comprise either a custom sequence used for annealing of the rolling circle replication primer, or a custom sequence used for hybridization of dye-labeled probe-specific detector-probe, or both. In some embodiments, these two said custom sequences are located on the same arm, or on different arms. In some embodiments, any of these two said sequences is split between left and right arm, so that its integrity and functionality is restored only upon the circularization of the linear polynucleotide formed at step (e).

In some embodiments, the ligation products of step (e) are products of double ligation, each comprising first and second ligation junctions.

In some embodiments, providing conditions of step (e) for intramolecular circularization of linear polynucleotide generated at step (d) comprises ligation of 5′ and 3′ ends of linear single-stranded polynucleotide with the aid of single-stranded DNA ligase, wherein under the conditions of ligation the 5′ end of linear polynucleotide and the 3′ end of the linear polynucleotide are ligated, thus, generating the product of ligation comprising continuous circular strand of nucleic acid. Exemplary single-stranded DNA ligases are CircLigase™, CircLigase II™ (Epicentre), and Thermophage Ligase (Prokaria).

In some embodiments, providing conditions of step (e) for intramolecular circularization of linear polynucleotide generated at step (d) comprises ligation of 5′ and 3′ ends of linear single-stranded polynucleotide with the aid of double-stranded DNA ligases, e.g. T4 DNA Ligase, and a splint oligonucleotide, comprising the oligonucleotide complementary to both 5′ end and 3′ end sequences of linear polynucleotide, so that both ends are hybridized to the splint oligonucleotide to generate double-stranded complex, wherein 5′ end of linear polynucleotide is positioned in juxtaposition to the 3′ end of linear polynucleotide, wherein under the conditions of ligation the 5′ end of linear polynucleotide and the 3′ end of the linear polynucleotide are ligated, thus, generating the product of ligation comprising continuous circular strand of nucleic acid.

In some embodiments, enriching single-stranded circles of step (f) comprises digesting uncircularized linear fragments with the aid of one or more exonucleases.

In some embodiments, the primer of step (g) comprises a hairpin primer or blocked-cleavable rhPCR (RNase H-dependent PCR, IDT) primer, or a hairpin primer with blocked-cleavable 3′ end, to enhance the specificity of the rolling circle replication at step (h).

In some embodiments, the methods of present disclosure comprise simultaneous detection of multiple targets (termed multiplexed detection, or multiplexing), wherein multiple target-specific probes are used and the target- or probe-specific detector-probes are differentially labeled with fluorescent dyes.

In some embodiments, for multiplexed detection the target-specific detector-probes comprise the probes labeled with fluorescent dyes according to “Multicolor Combinatorial Probe Coding” (MCPC) (Qiuying Huang, et al. (2011), PLoS ONE Volume 6, Issue 1, e16033), the labeling paradigm, which uses a limited number (n) of differently colored fluorophores in various combinations to label each probe, enabling all of 2ⁿ-1 targets to be detected in one reaction.

Provided herein also are the methods and compositions (FIG. 9B) as applied to non-invasive prenatal testing for detection of fetal aneuploidies, comprising detection and quantification of chromosome-specific or locus-specific target polynucleotides, wherein each of multiple probe sets and their corresponding detector probes are specific for single chromosome or chromosome locus, wherein the chromosomes are selected from the list of chromosomes susceptible to aneuploidy and reference chromosomes. The method of prenatal testing for detection of fetal aneuploidies, said method comprising: providing a sample comprising plurality of polynucleotide fragments, wherein the polynucleotide fragments comprise fetal and maternal cell-free genomic DNA, providing denaturing conditions under which the polynucleotide fragments are converted to single-stranded form, contacting the sample with plurality of chromosome-specific probes, wherein probes specifically anneal to the complementary sequences of the target chromosomes to form a double stranded complex, wherein said plurality of chromosome-specific probes comprises at least 100 different polynucleotide sequences selected from a first chromosome tested for being aneuploidy (probe-set for the first chromosome), and at least 100 different polynucleotide sequences selected from a reference chromosome (probe-set for the reference chromosome), wherein the first chromosome tested for being aneuploid and the reference chromosome are different, providing conditions for hybridization and ligation, under which said conditions the plurality of chromosome-specific probes hybridizes to its target sequences within nucleic acid fragments and generates the plurality of chromosome-specific ligation products, each ligation product being a linear polynucleotide comprising at least one ligation junction, providing conditions for intramolecular circularization of plurality of linear polynucleotides, comprising conditions for ligation of the free ends of said linear polynucleotides, to result in formation of plurality of chromosome-specific single-stranded circles, enriching single-stranded circles by enzymatically digesting un-circularized linear nucleic acids, contacting the single-stranded circles with the plurality of chromosome-specific oligonucleotide primers, comprising annealing conditions under which the primers hybridizes to the complementary sequences of the chromosome-specific circles to form a double stranded primer-circle complexes suitable for initiation of DNA synthesis, wherein said plurality comprises at least one primer specific for circles derived from the first chromosome tested for being aneuploidy, and at least one primer specific for circles derived from the reference chromosome, providing conditions for rolling circle replication of the chromosome-specific primer-circle complexes, wherein the circles derived from the first chromosome tested for being aneuploid, and the circles derived from the reference chromosome are selectively amplified to generate long linear single-stranded products, detecting the product of rolling circle replication, comprising depositing of said product on solid phase, hybridizing said product with plurality of chromosome-specific dye-labeled detector-probes, and imaging, wherein said plurality of chromosome-specific detector-probes comprises at least one polynucleotide sequence complementary to the custom sequence of chromosome-specific probe set of step (c) selected from a first chromosome tested for being aneuploid, and at least one polynucleotide sequence complementary to the custom sequence of chromosome-specific probe set of step (c) selected from a reference chromosome, wherein the first chromosome tested for being aneuploid and the reference chromosome are different, wherein the plurality of detector-probes specific to the first chromosome tested for being aneuploid labeled with the same fluorescent dye, which is different from the fluorescent dye of plurality of detector-probes specific for the reference chromosome, wherein detecting of said product indicates the presence of the target polynucleotide in the sample, enumerating the products exhibiting dye-specific signal, comprising enumerating the rolling circle replication products corresponding to fetal and maternal polynucleotide fragments specific for the first chromosome tested for being aneuploid and products specific for the reference chromosome, determining for the cell-free DNA sample the presence or absence of a fetal aneuploidy comprising using a number of enumerated rolling circle replication products corresponding to the first chromosome and a number of enumerated rolling circle replication products corresponding to the reference chromosome of (j).

In some embodiments, the first chromosome tested for being aneuploid is selected from the group consisting of chromosome 13, chromoomem18, chromosome 21, chromosome X, and chromosome Y; and the reference chromosome is selected from the group consisting of chromosome 1, chromosome 2, and chromosome 3.

In some embodiments, the method interrogates major aneuploidies detected in human population comprising trisomy 21, trisomy 18, trisomy 13, and monosomy X.

In another aspect, method (FIG. 9C) of detection of target nucleic acid in the sample using circularization of nucleic acid fragments of the sample is provided, said method comprising: providing a sample comprising plurality of polynucleotide fragments, providing conditions for end-repair of polynucleotide fragments comprising restoration of 5′ phosphate and 3′ hydroxyl groups, providing denaturing conditions under which the polynucleotide fragments are converted to single-stranded form, providing conditions for intra-molecular circularization of polynucleotide fragments, comprising ligase and cofactors, to result in formation of single-stranded circles, enriching a single-stranded circles by enzymatically digesting un-circularized linear fragments, contacting the single-stranded circles with at least one target-specific oligonucleotide comprising the primer of deoxyribonucleic acid synthesis, comprising annealing conditions under which the primer hybridizes to the complementary sequence of the circular target polynucleotide to form a double stranded complex, providing conditions for rolling circle replication of the target-specific primer-circle complex, detecting the product of rolling circle replication, comprising depositing of said product on solid phase, hybridizing of said product with target-specific dye-labeled detector-probe, and imaging, wherein detecting of said product indicates the presence of the target polynucleotide in the sample, and enumerating the products with detected dye-specific signal, and correlating the detected product of rolling circle replication with an amount of the target polynucleotide sequence present in the sample.

In some embodiments, a sample of (a) comprises fragmented deoxyribonucleic or ribonucleic acids. In some embodiments, the nucleic acid fragments comprise various species of viral, bacterial, yeast, fungal, and higher eukaryote DNA and RNA.

In some embodiments, the fragments of ribonucleic acids are converted to the deoxyribonucleic acid by the process of reverse transcription.

In some embodiments, providing conditions for end-repair of polynucleotide fragments of step (b) comprises the treatment of a sample with polynucleotide kinase.

In some embodiments, providing conditions of step (d) for intra-molecular circularization of polynucleotide fragments comprises ligation of 5′ and 3′ ends of linear single-stranded polynucleotide fragment with the aid of single-stranded DNA ligase, wherein under the conditions of ligation the 5′ end of linear polynucleotide and the 3′ end of the linear polynucleotide are ligated, thus, generating the product of ligation comprising continuous circular strand of nucleic acid. Exemplary ssDNA ligases are CircLigase™, CircLigase II™ (Epicentre), and Thermophage Ligase (Prokaria).

In some embodiments, the primer of step (f) comprises a hairpin primer or blocked-cleavable rhPCR (RNase H-dependent PCR) primer, or a hairpin primer with blocked-cleavable 3′ end, to enhance the specificity of the rolling circle replication at step (g).

In some embodiments, the methods of present disclosure comprise simultaneous detection of multiple targets (termed multiplexed detection, or multiplexing), wherein the target-specific detector-probes are labeled with distinguishable fluorescent dyes.

In some embodiments, for multiplexed detection the target-specific detector-probes comprise the probes labeled with fluorescent dyes according to “Multicolor Combinatorial Probe Coding” (MCPC)(Qiuying Huang, et al. (2011), PLoS ONE Volume 6, Issue 1, e16033), the labeling paradigm, which uses a limited number (n) of differently colored fluorophores in various combinations to label each probe, enabling all of 2ⁿ-1 targets to be detected in one reaction.

In some embodiments, the step (g) comprises a rolling circle replication in the presence of unlabeled dNTPs, or the fluorescently-labeled dNTP, or combination of both at specific ratio. In some embodiments, detecting the product of step (h) comprises depositing of said product on solid phase and imaging the fluorescence.

In some embodiments, the step (g) comprises a pulse-chase reaction, wherein during the time course of rolling circle replication in the presence of unlabeled dNTPs the fluorescently-labeled dNTP is added at specific time point and reaction continues until the replication products became fluorescently labelled.

In some embodiments, the multiplexed detection comprise the splitting of the sample in two or more parts after any of the steps of the method, from step (a) through step (e), and performing the detection of each target nucleic acid, present in the sample, separately. In some embodiments, the mutiplexing comprises the splitting of the sample and performing the rolling circle replication separately for each target in dedicated compartment, wherein said rolling circle replication is performed in the presence of unlabeled dNTPs mixed with fluorescently-labeled dNTP at specific ratio, wherein differently colored fluorophores are attached to different dNTPs, and wherein various combinations of limited number of differently colored dNTPs and various ratios between unlabeled and fluorescently-labeled dNTPs are used to achieve higher level of multiplexing than the number of colored fluorophores. In some embodiments, the products of rolling circle replication are pooled together prior to their deposition on the solid substrate for detection and de-multiplexing.

In another aspect, method (FIG. 9D) of detection of chromosomal aneuploidy in a sample comprising fetal and maternal cell-free genomic DNA, said method comprising: providing a sample comprising plurality of polynucleotide fragments, wherein the polynucleotide fragments comprise fetal and maternal cell-free genomic DNA, providing conditions for end-repair of polynucleotide fragments comprising restoration of 5′ phosphate and 3′ hydroxyl groups, providing denaturing conditions under which the polynucleotide fragments are converted to single-stranded form, providing conditions for intramolecular circularization of polynucleotide fragments, comprising ligase and cofactors, to result in formation of single-stranded circles, enriching a single-stranded circles by enzymatically digesting uncircularized linear fragments, contacting the single-stranded circles with plurality of chromosome-specific oligonucleotides comprising the primers of DNA synthesis, under conditions wherein the primers specifically hybridize to the complementary sequences of the circles to form a double stranded primer-circle complexes, wherein said plurality of chromosome-specific primers comprise at least 100 different polynucleotide sequences selected from a first chromosome tested for being aneuploid, and at least 100 different polynucleotide sequences selected from a reference chromosome, wherein the first chromosome tested for being aneuploid and the reference chromosome are different, providing conditions for rolling circle replication of the chromosome-specific primer-circle complexes, wherein the circles derived from the first chromosome tested for being aneuploid, and the circles derived from the reference chromosome are selectively amplified to generate long linear single-stranded products each comprising at least 200 copies of circularized target chromosome fragment, detecting the products of rolling circle replication, comprising depositing of said products on solid phase, hybridizing said products with plurality of target-specific dye-labeled detector-probe, and imaging, wherein said plurality of target-specific detector-probes comprises at least 100 different oligonucleotide sequences fully or partially complementary to the primers of step (f) selected from a first chromosome tested for being aneuploid, and at least 100 different oligonucleotide sequences complementary to the primers of step (f) selected from a reference chromosome, wherein the number of detector-probes is equal to the number of chromosome-specific primers of step (f), wherein the plurality of detector-probes specific to the first chromosome tested for being aneuploid is labeled with the same fluorescent dye, which is different from the fluorescent dye used for labeling of plurality of detector-probes specific for the reference chromosome, enumerating the products exhibiting dye-specific signal, comprising enumerating the rolling circle replication products corresponding to fetal and maternal polynucleotide fragments specific for the first chromosome tested for being aneuploid and products specific for the reference chromosome, determining for the cell-free DNA sample the presence or absence of a fetal aneuploidy, comprising using a number of enumerated rolling circle replication products corresponding to the first chromosome and a number of enumerated rolling circle replication products corresponding to the reference chromosome of (i).

In some embodiments, the first chromosome tested for being aneuploid is selected from the group consisting of chromosome 13, chromoomem18, chromosome 21, chromosome X, and chromosome Y; and the reference chromosome is selected from the group consisting of chromosome 1, chromosome 2, and chromosome 3.

In some embodiments, the method interrogate major aneuploidies detected in human population comprising trisomy 21, trisomy 18, trisomy 13, and monosomy X.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

### Part A—Methods of Detecting Target Nucleic Acids Using a Positively Charged Tag

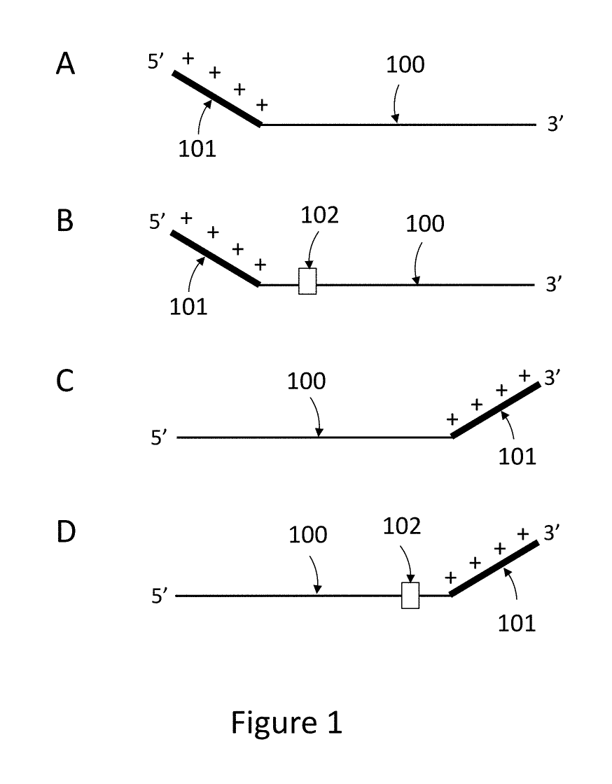

FIG. 1 is a schematic showing various probe configurations. The probe comprises a target complementary segment and a detectable tag, or flap. In Panels (A) and (B), the tag is at the 5′ end of the probe; in Panels (C) and (D), the tag is at the 3′ end of the probe. In Panels (B) and (D), the probe contains a scissile linkage adjacent to the tag.

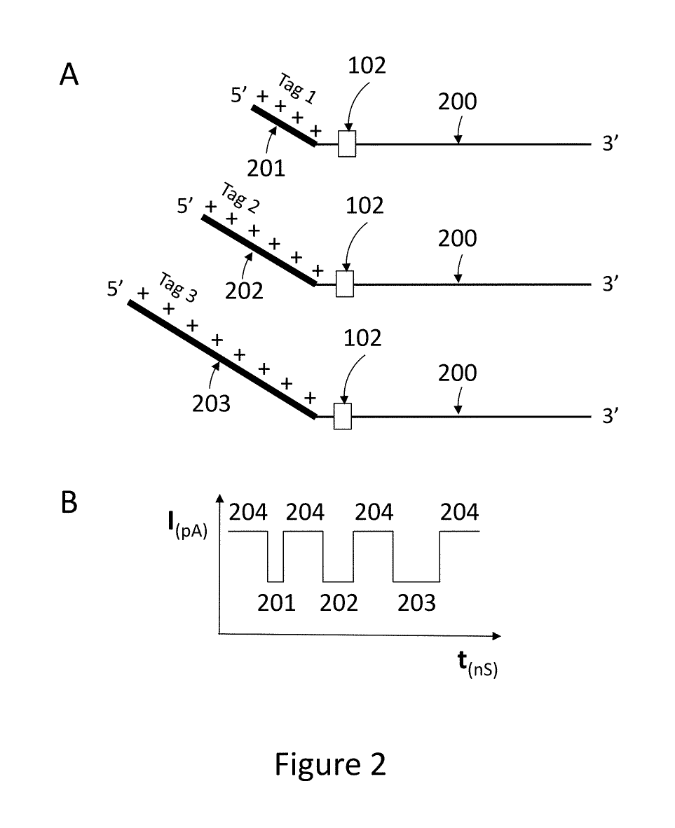

FIG. 2 is a schematic of probes used for multiplexing. Panel (A) depicts an example of probes containing detectable tags with different number of positive charges. Panel (B) shows an example of the electrical signal generated by such multiplexed probes.

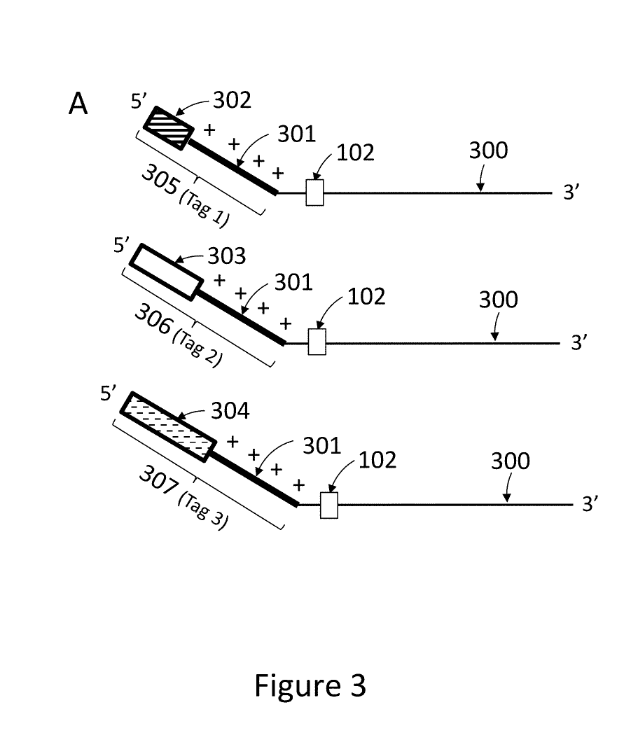

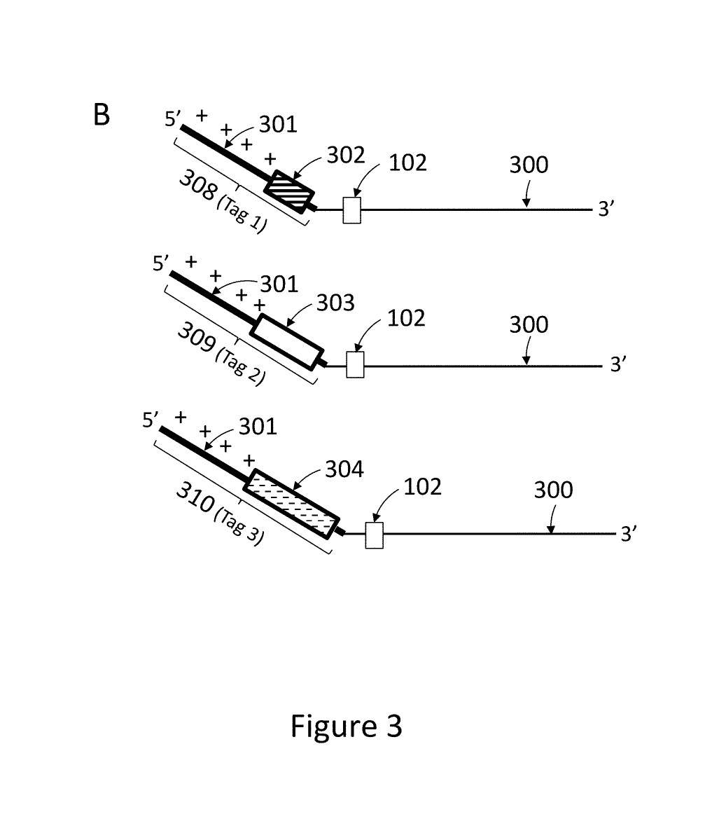

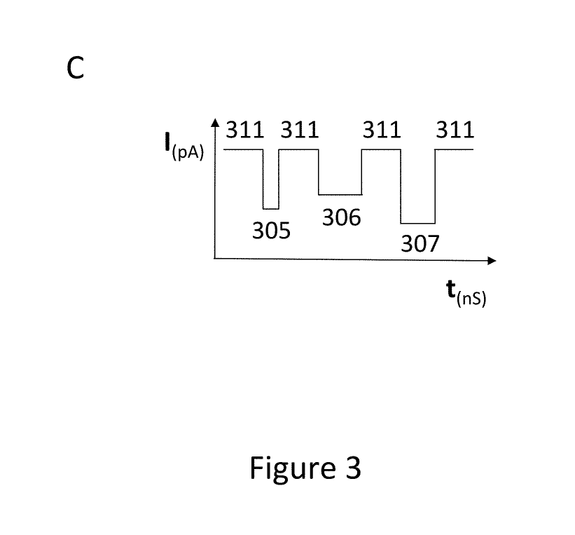

FIG. 3 is a schematic of another approach for multiplexing. Panels (A) and (B) depict an example of probes with the detectable tags comprising positively charged moieties 301 and additional chemical compounds (neutral or positively charged). Panel (C) depicts an example of electrical signal produced by these probes while flowing through the nanopore.

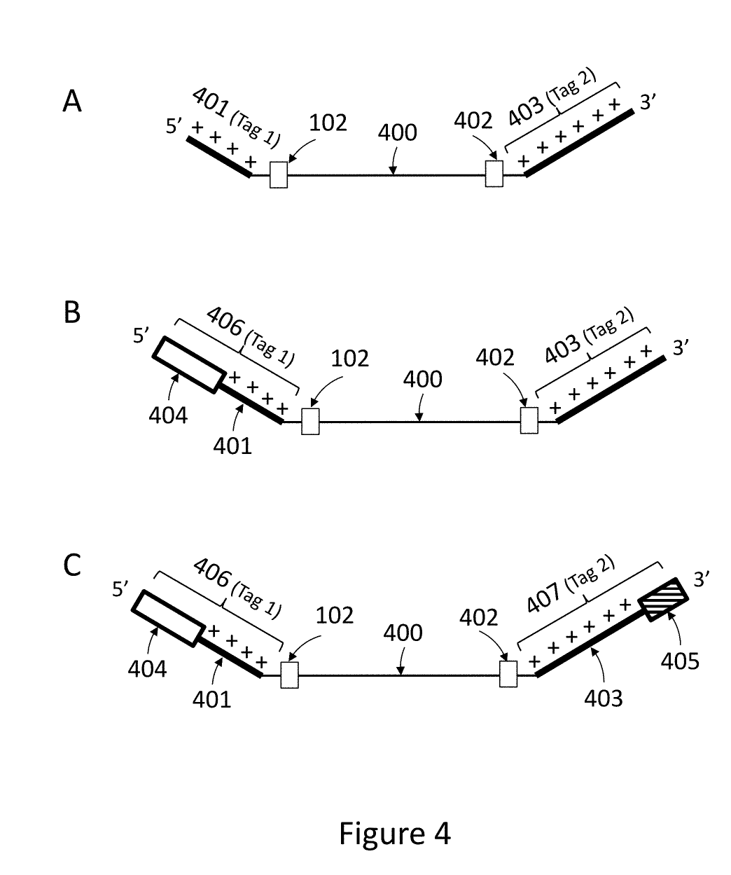

FIG. 4 is a schematic of probes having dual tags. Panels (A), (B) and (C) depict examples of probes with 5′ and 3′ detectable tags, or flaps. In the embodiments shown in Panels (A), (B) and (C), the tags include various combinations of positively charged moieties and additional chemical compounds (neutral or positively charged).

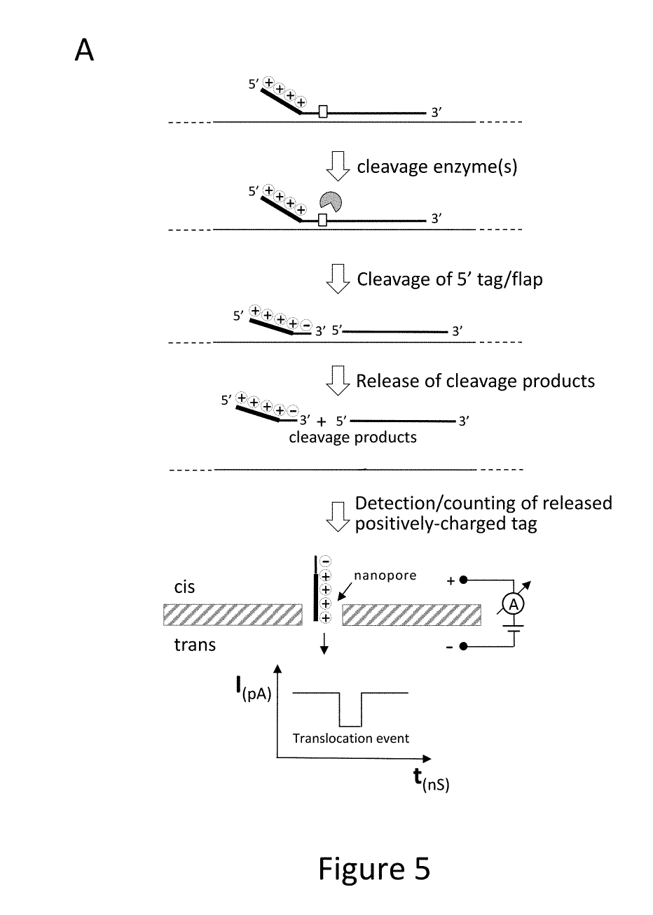

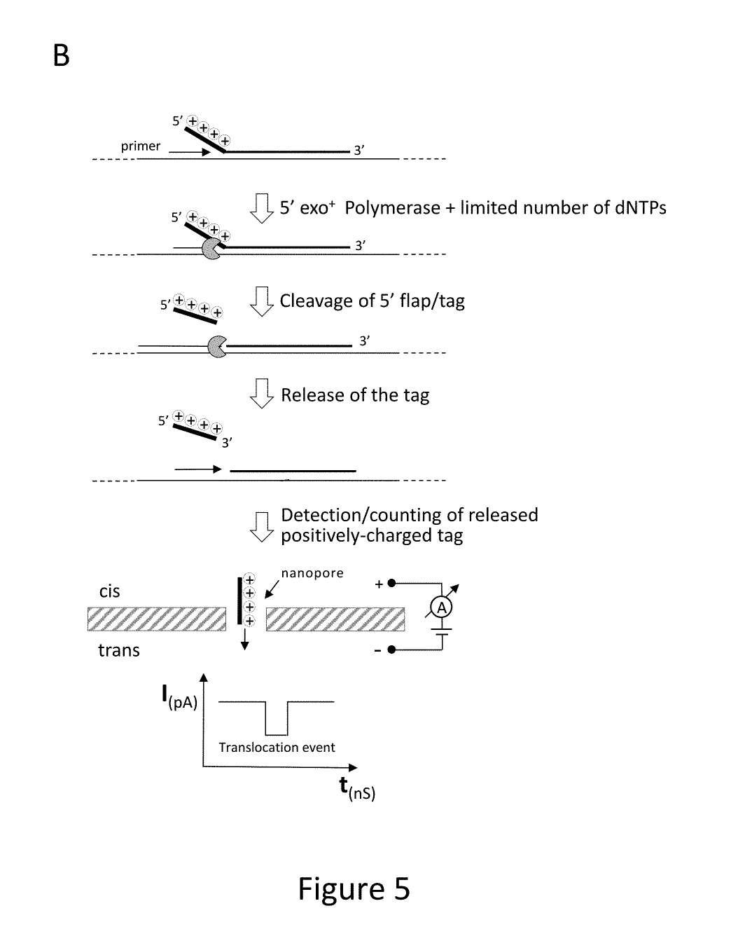

FIG. 5 is a schematic overview of an exemplary method for direct (i.e., amplification-free) electronic detection of target nucleic acid using a nanopore detector. Panel (A) shows that the probe anneals to the target region and is cleaved by the enzyme (e.g., endonuclease or lyase) that is specific for the scissile linkage (modification) to release the positively charged tag (or flap). The released tag is subjected to electrical field force and translocates through the nanopore to generate the electrical signal, e.g. the change in conductance. Other nucleic acid species are negatively charged and remain in the cis-compartment. Panel (B) shows that the probe and the upstream primer anneals to the target polynucleotide in a tandem separated by a nick or a 1 nt gap. A DNA polymerase having 5′-exonuclease activity performs limited DNA synthesis; the detectable 5′ tag is cleaved by the DNA polymerase/exonuclease, released, and detected as described herein.

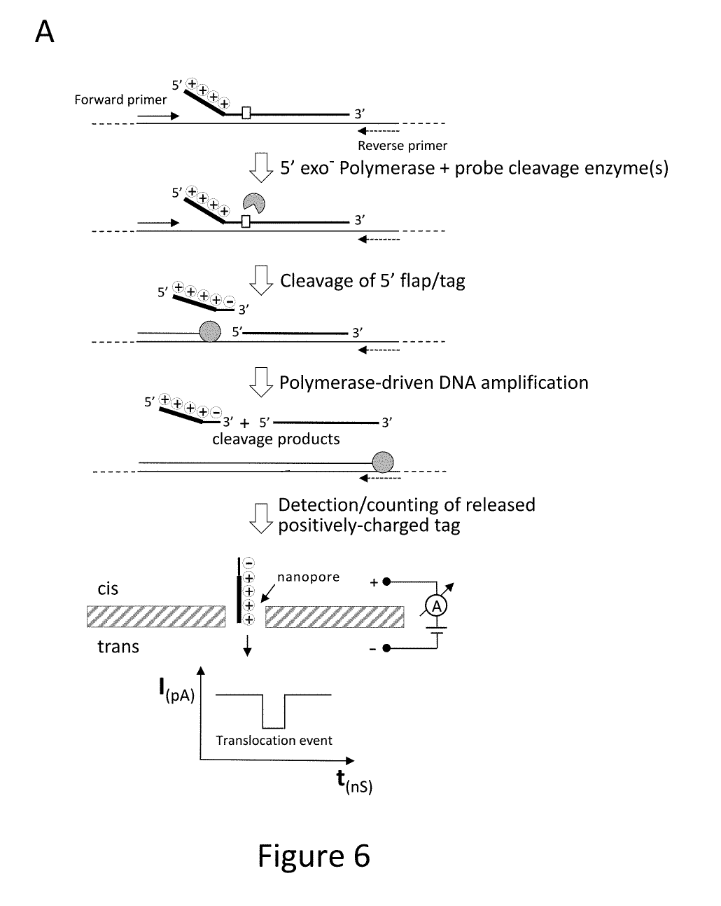

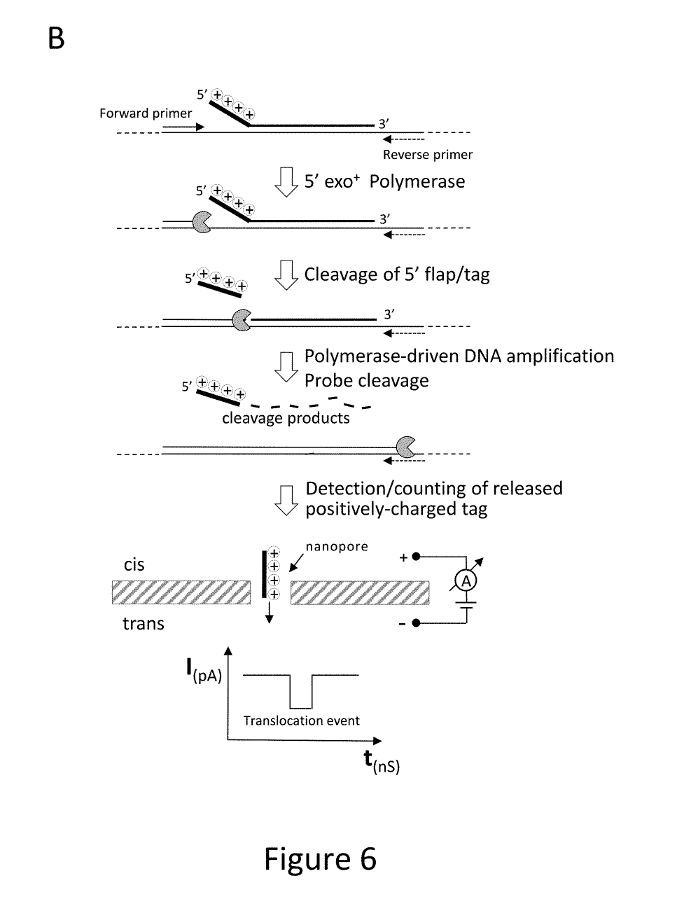

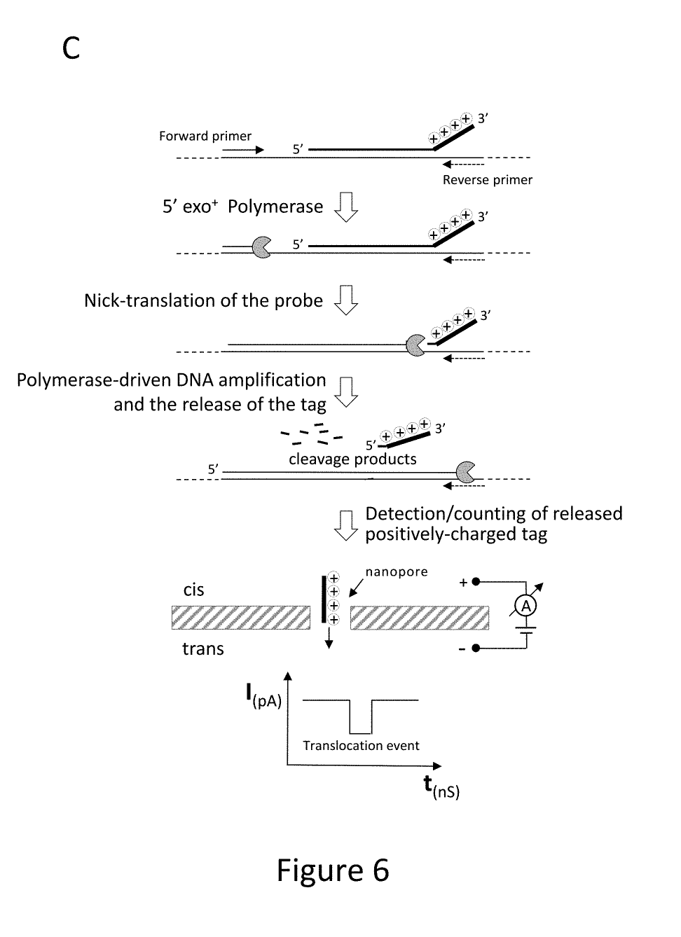

FIG. 6 is a schematic overview of exemplary methods for amplification-based electronic detection of target nucleic acid using a nanopore detector. The target DNA is amplified using forward and reverse primers and a probe. During amplification, the probe anneals to the target amplicon region and is cleaved to release the detectable positively charged tag (or flap). The released tag is subjected to electrical field force and translocates through the nanopore to generate the electrical signal, e.g. the change in conductance. Other nucleic acid species are negatively charged and remain in cis-compartment. Panel (A) is a schematic showing the use of a strand-displacing polymerase, which lacks 5′ exonuclease activity. In the course of amplification, the probe having the scissile linkage hybridizes to the target and is cleaved by the enzyme (e.g., endonuclease or lyase) that is specific for the scissile linkage (modification); the positively-charged detectable tag is released, together with the displaced target complementary portion of the probe bearing the negative charge. Panel (B) is a schematic showing the use of a DNA polymerase having 5′ exonuclease activity. During amplification, the tag (or flap) located at the 5′ end of the probe is cleaved off by the activity of the 5′-3′ exonuclease (also called a flap endonuclease); the rest of the probe is degraded by the same activity. Panel (C) is a schematic showing the use of a DNA polymerase having 5′ exonuclease activity as in Panel (B), but the detectable tag in Panel (C) is located at the 3′ end of the probe. DNA polymerase nick-translates the target complementary portion of the probe until the detectable tag (or flap) is released.

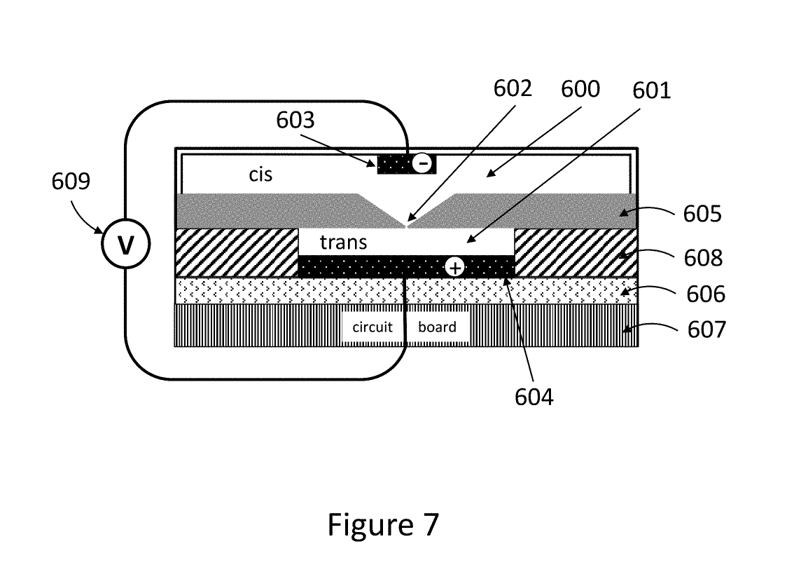

FIG. 7 is a schematic showing one example of a device with an integrated nanopore detector. The device features a reverse polarity of electrodes relative to conventional DNA sequencing nanopore devices, as, in the present case, the positively charged tag is detected by translocation through the nanopore.

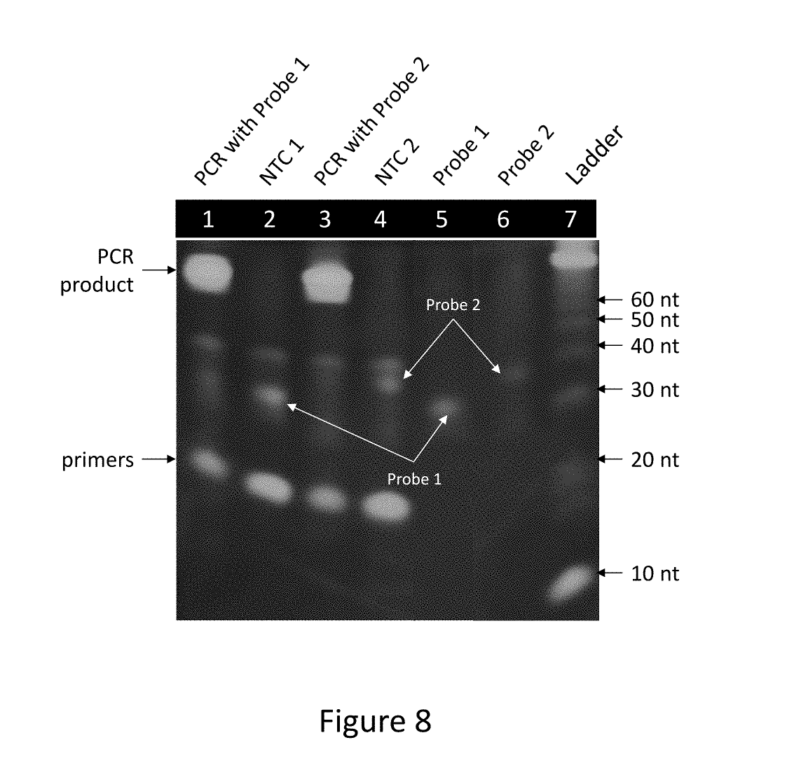

FIG. 8 is a photograph of an electrophoretic gel. The gel shows an example of target DNA amplification by PCR with concomitant cleavage of the probes, which include positively charged tags. The probe-bearing tags at the 5′ or 3′ ends are both cleaved by Taq DNA polymerase.

### Part B—Methods of Detecting Target Nucleic Acids Using Rolling Circle Replication

FIG. 9 is a flow chart of the methods described herein for digital detection and quantification of nucleic acid target molecules. The methods in Panels (A) and (B) involve the rolling circle replication of the circularized probe. The methods in Panels (C) and (D) involve the rolling circle replication of the circularized fragments of DNA having a target sequence. The methods in Panels (B) and (D) involve prenatal testing for chromosomal aneuploidy using samples containing circulating, cell free DNA.

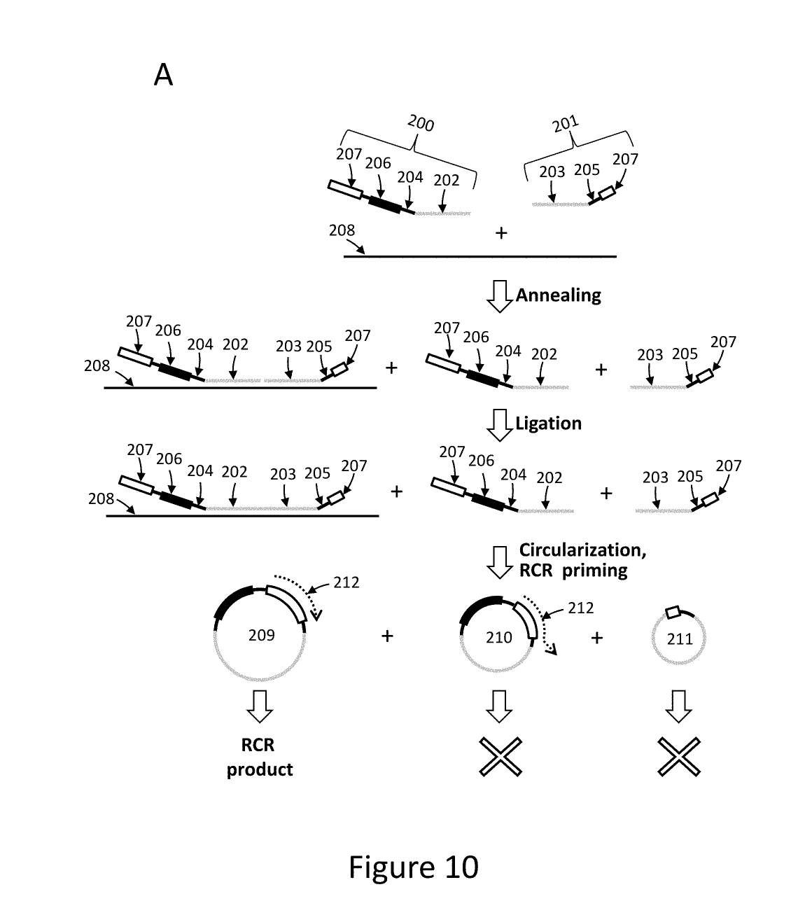

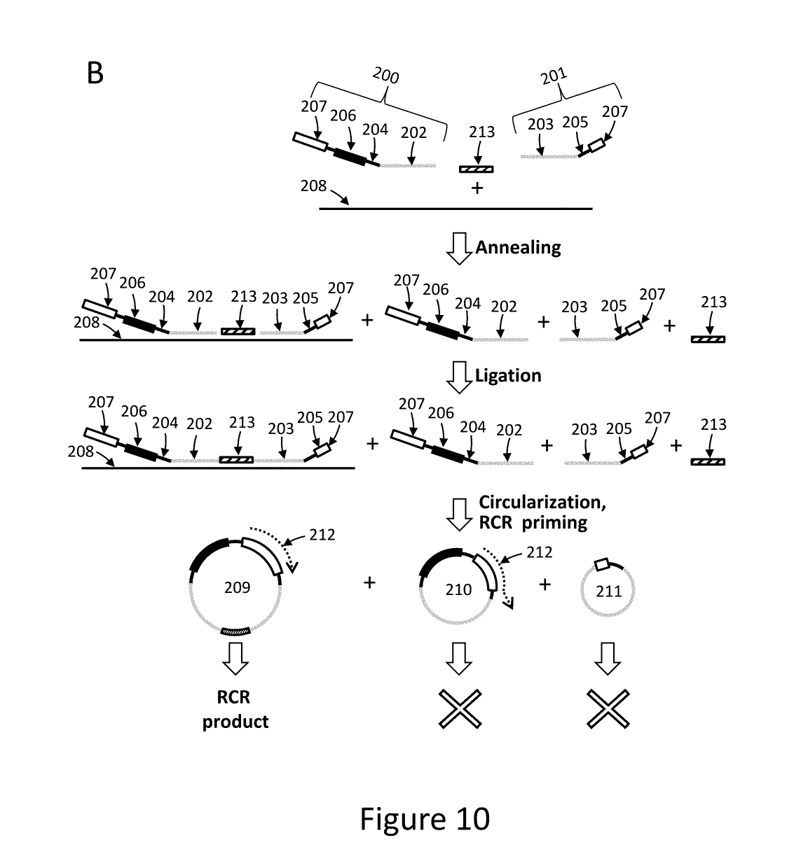

FIG. 10 are schematics of the probe and the method of probe circularization. Panel (A) depicts an example of a probe and probe circularization using a single-stranded DNA ligase. Panel (B) is similar to Panel (A), but the probe in Panel (B) includes a “bridge” oligonucleotide.

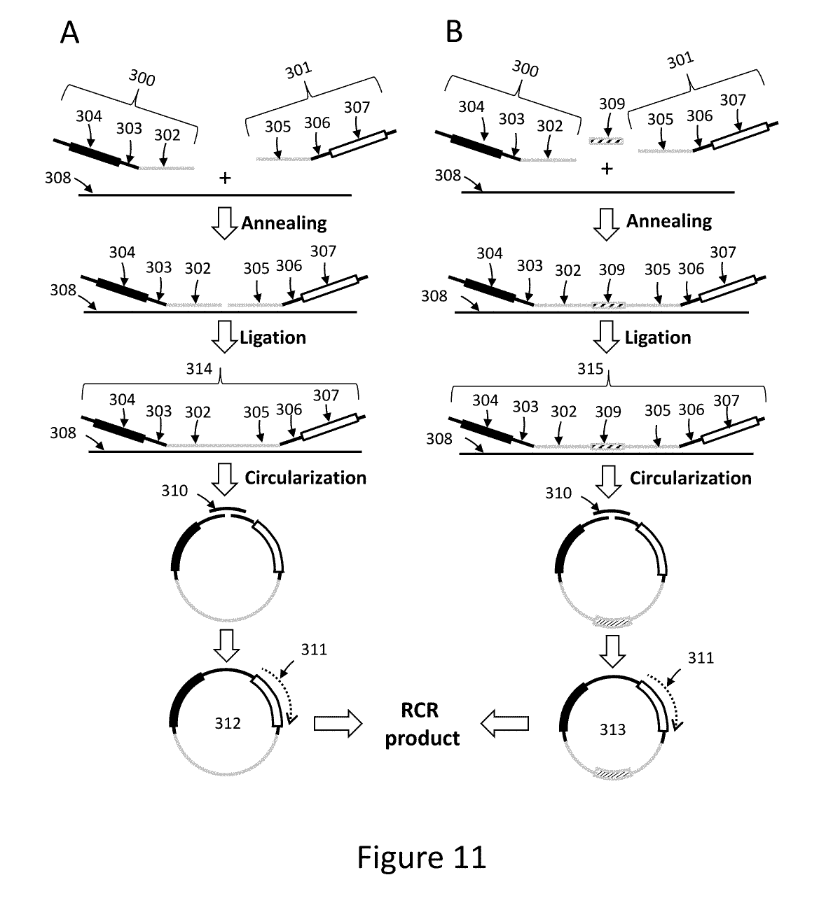

FIG. 11 is a schematic of the probe and the method of probe circularization. Panel (A) depicts an example of a probe and probe circularization using a double-stranded DNA ligase and a “splint” oligonucleotide as a ligation template. Panel (B) is similar to Panel (A), but the probe in Panel (B) includes a “bridge” oligonucleotide.

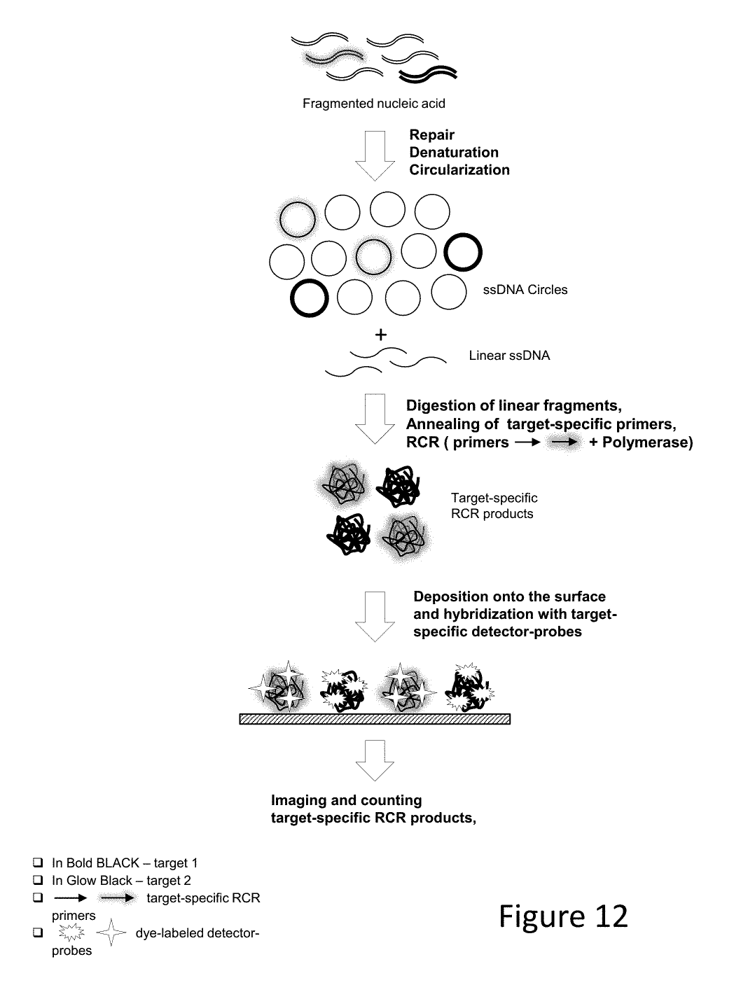

FIG. 12 is a schematic overview of a method for detecting target nucleic acid, which includes the circularization of nucleic acid fragments of the sample. As an example, the method for detecting two targets is depicted, where the products of rolling circle replication are visualized upon hybridization with the target-specific dye-labeled detector-probes.

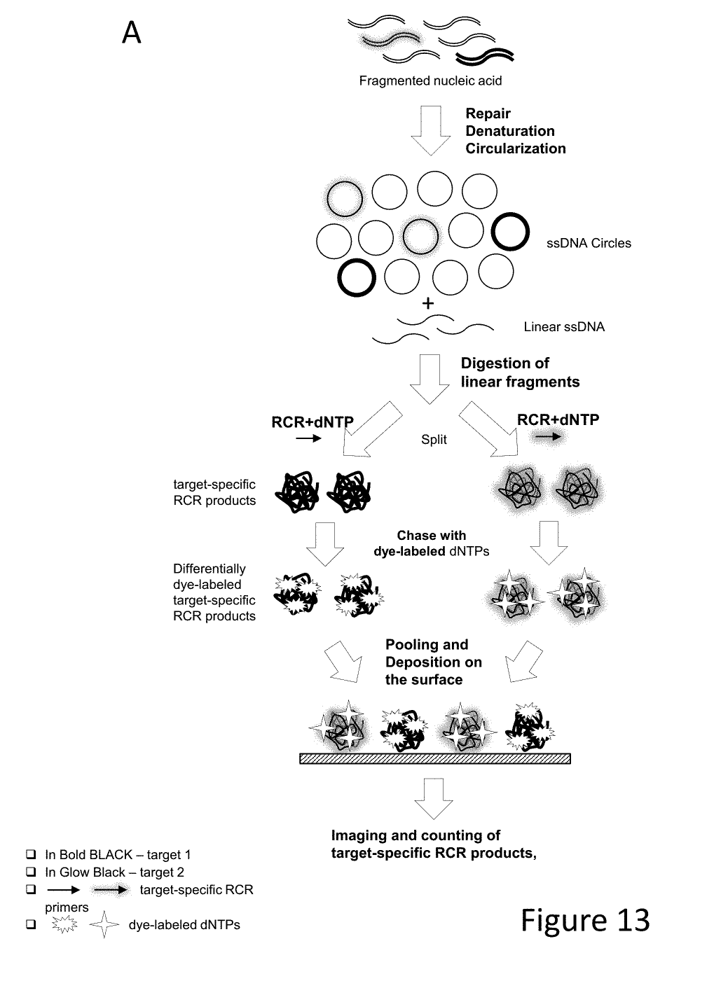

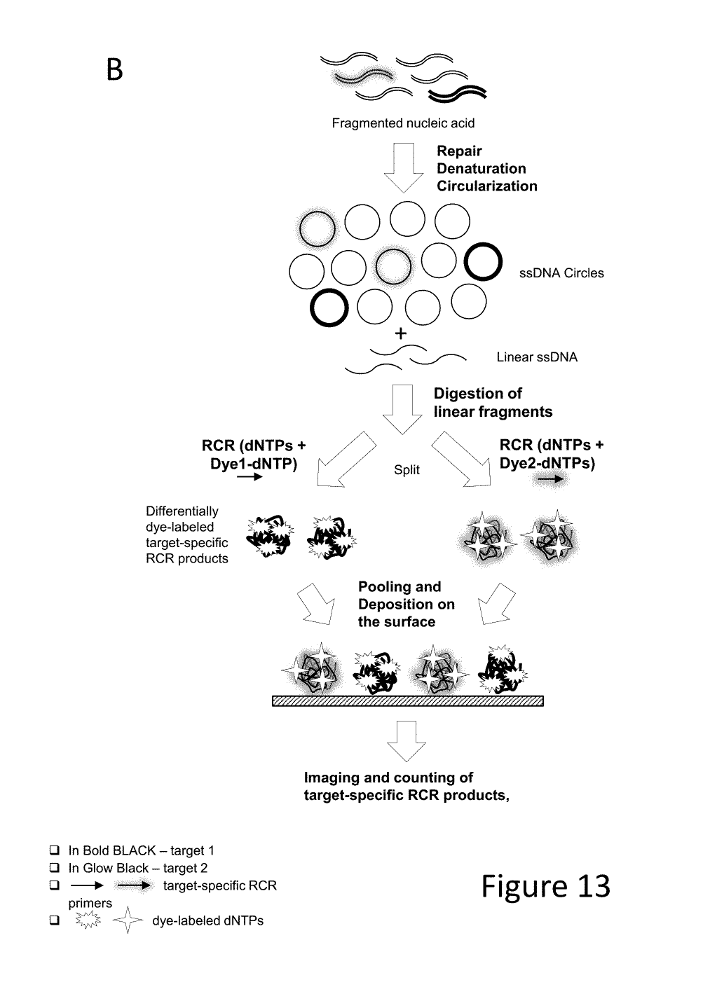

FIG. 13 is a schematic overview of a method for detecting target nucleic acid, which includes the circularization of nucleic acid fragments of the sample. As an example, the method for detecting two targets is depicted, where the products of rolling circle replication are distinguishably labeled with the fluorescently-labeled dNTPs added to the RCR reaction at the late stage (Panel (A)), or as an admixture with the unlabeled dNTPs at the reaction initiation (Panel (B)).

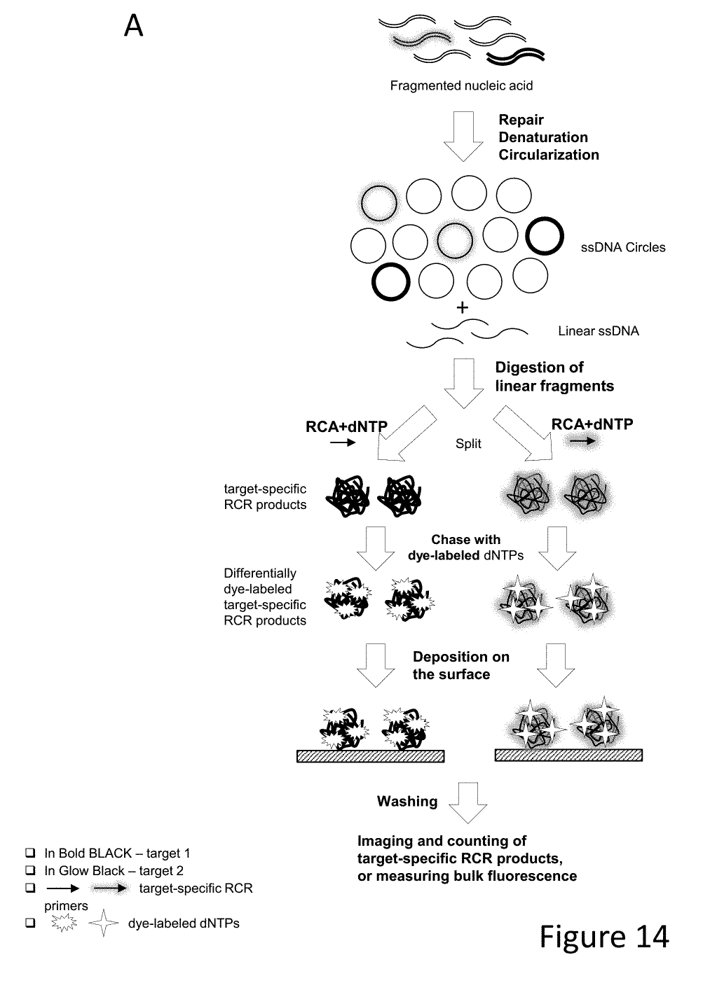

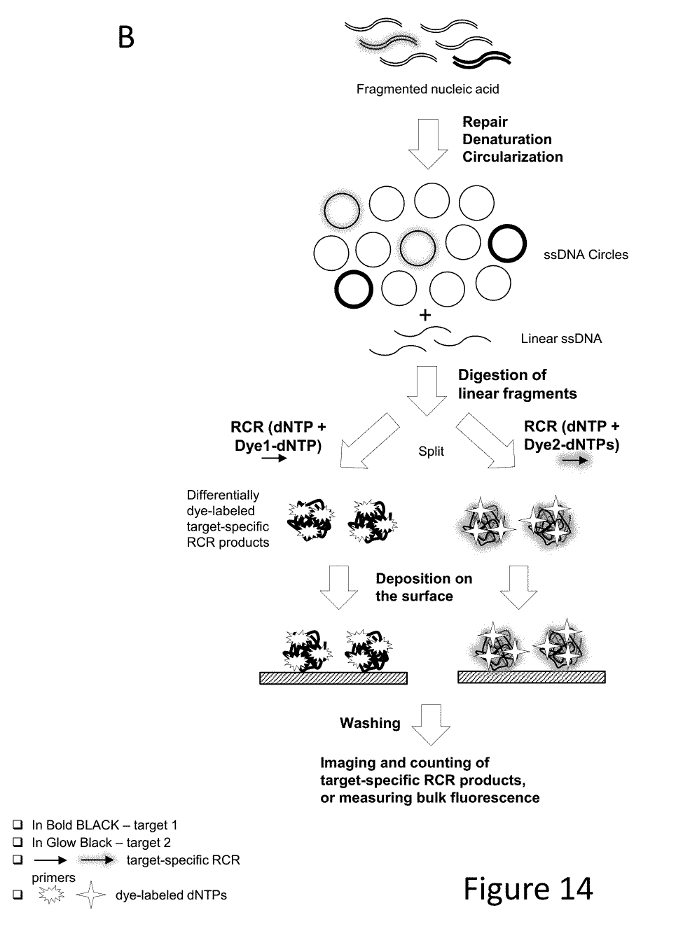

FIG. 14 is a schematic overview of a method for detecting target nucleic acid, which includes the circularization of nucleic acid fragments of the sample. As an example, the method for detecting two targets is depicted, where the products of rolling circle replication are distinguishably labeled with the fluorescently-labeled dNTPs added to the RCR reaction at the late stage (Panel (A)), or as an admixture with the unlabeled dNTPs at the reaction initiation (Panel (B)). The sample including the single-stranded circle intermediates is split, and both the rolling circle replication and detection are performed in separate compartments.

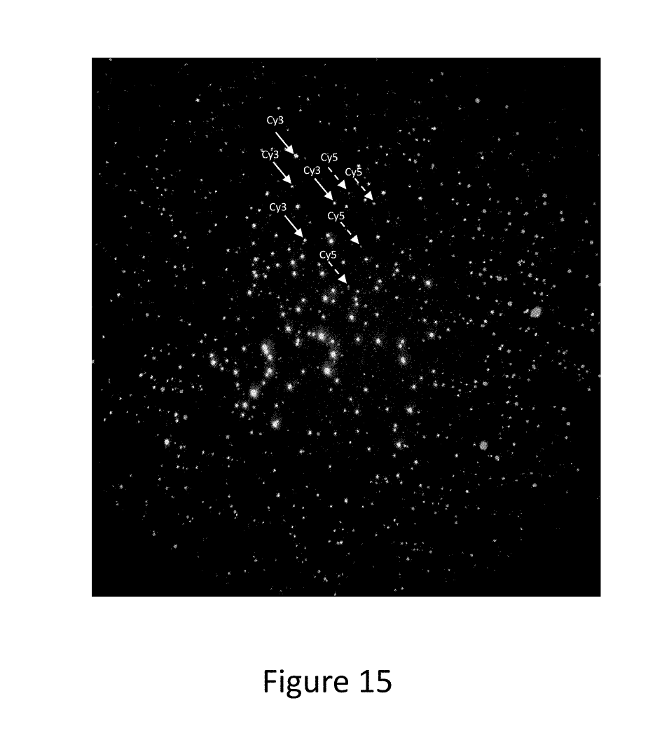

FIG. 15 is a composite image of Cy3 and Cy 5 fluorescence of the same field of view taken by fluorescent microscopy showing the detection of RCR products specific for human Chromosome 1 and 21 correspondingly after deposition of their mix on the surface of the amino-silanated glass slide.

### DETAILED DESCRIPTION

In the last two decades many methods of target nucleic acid detection have been developed, and some are currently widely used in medical, agricultural, bio-safety, and food-safety applications. Among them the isothermal target nucleic acid detection methods (e.g. NASBA, LAMP, and RPA) and PCR are the most common. The PCR-based methods, especially the probe-based PCR, like TaqMan assay, are considered to be a “gold standard” due to their robustness, simplicity, sensitivity, and specificity. However, their output is an analog optical signal generated by fluorescently labeled probes or primers, and intercalating dyes.

Recently, droplet digital PCR (ddPCR) technologies (Bio-Rad, Inc., RainDance Technologies, Inc), often called next generation PCR, were developed to provide the capability of absolute quantification of the target nucleic acid in a sample. However, these technologies require a fluidic device to generate emulsion droplets in order to compartmentalize single molecules, in addition to the PCR module. Moreover, there are certain limitations of the method related to PCR biases, limited amount of reagents in very small reactors, and non-uniformity in droplet size and composition. Additionally, the emulsion droplet technology precludes multistep chemistries where reagents are added incrementally, reducing its overall utility.

Non-invasive prenatal testing (NIPT) using cell-free fetal (cff) DNA circulating in maternal blood allows for earlier detection of genetic diseases and common chromosomal aneuploidies such as, e.g., Trisomies 13, 18, and 21. This helps to make decisions on reproductive health and pregnancy management. Commercialized NIPT assays are based on massively parallel PCR amplification of regions of affected chromosomes followed by detection of chromosome imbalances in fetal genome by shotgun sequencing or microarray techniques. There is a need to improve cost and turnaround time for NIPT. The present disclosure provides methods and compositions for PCR-free and sequencing-free NIPT.

These methods represent novel approaches to detect nucleic acid targets and can be employed for other applications, e.g., pathogen detection, genotyping, etc.

### Part A—Methods of Detecting Target Nucleic Acids Using a Positively Charged Tag

### 1. Overview

Methods of electronically detecting target nucleic acids are provided herein. The target nucleic acids can be amplified (e.g., using, without limitation, PCR or isothermic amplification), but the methods described herein do not require amplification (i.e., the methods described herein include amplification-free methods). One of the features of the methods described herein is a probe, which serves as a source of a detectable positively-charged tag that is released upon hybridization with the complementary target nucleic acid. When the target polynucleotide is amplified with the aid of specific amplification primers, hybridization of the probe to the amplicon results in the amplification-dependent cleavage of the probe, thus, additionally increasing the specificity and sensitivity of the detection of the target nucleic acid.

In addition to the region of complementarity to the target nucleic acid, which is negatively charged, the probe includes a detectable positively-charged tag, which forms a flap upon hybridization of the probe to the target nucleic acid. The target-dependent release of the tag from the probe by the cleavage action of an enzyme can be electronically detected using a nanopore (e.g., upon translocation of a tag through the nanopore), which is different from other methods of probe-based nucleic acid detection methods including optical, electrochemical, optomechanical, and electromechanical methods. A schematic flow chart of the exemplary methods of present disclosure is shown in FIGS. 5 and 6.

As part of the methods described herein, a system for detecting target nucleic acids using a positively charged tag also is provided. Such a system typically includes a microfluidic device having a nanopore sensor. Such a microfluidic device typically includes a first compartment containing a (+) electrode and the sample in conductive buffer solution, a second compartment containing a (−) electrode, and a nanopore embedded in the barrier separating the two compartments. Such a microfluidic device also can include a temperature control element, a circuit board with an amplifier coupled to a computer processor (including, e.g., memory) and signal processing software. It would be appreciated that the methods and systems described herein can allow for real-time detection of target nucleic acids. Importantly, the disclosed microfluidic device is portable, miniaturized (e.g., about the size of a “memory stick” or a “jump drive”), which makes it ideal for point-of-care as well as field applications.

Also as part of the methods and systems described herein, a kit is provided that can be used to detect target nucleic acids. A kit as described herein can include one or more probes specific for a single target or a plurality of targets (e.g., multiplexed target detection). A kit as described herein also can include one or more amplification primers, buffers, and enzymes. In addition, a kit also can include a microfluidic device as described herein.

Methods of electronically detecting target nucleic acids as described herein can be used for detecting various targets of medical, agricultural, biodefence, food safety, and environmental monitoring significance. These include, without limitation, various pathogens (e.g., bacterial or viral), single nucleotide polymorphism (SNP) variants, mutations, or genome structural variations.

The methods and systems described herein possess multiple features that can significantly reduce the costs of in vitro diagnostics. For example, the methods and systems described herein do not require expensive optical components, as with other currently used devices. Therefore, the low cost of the microfluidic device as described herein allows it to be constructed as a single-use disposable device, in contrast to currently used bulky optical detection devices.

### 2. Methods and Systems for Nucleic Acid Detection

Existing strategies for nucleic acid detection can be grouped into three categories: target amplification, probe amplification, and signal amplification. A number of methods have been developed for target amplification, with polymerase chain reaction (PCR) currently being the gold standard for various diagnostic applications. Quantitative PCR methods using read out fluorescent probes, e.g., TaqMan dual-labeled probes, Scorpion probes, Molecular Beacons, LightCycler probes, and intercalating dyes (e.g.SYBR Green), are the most common methods in nucleic acid diagnostics, but requires sophisticated and expensive equipment to perform thermal cycling and optical readout. To circumvent limitations in PCR, especially in a point-of-care setting, various isothermal amplification techniques has been developed including strand displacement amplification (SDA), loop-mediated amplification (LAMP), nucleic acid sequence-based amplification (NASBA), helicase-dependent amplification (HDA), recombinase polymerase amplification (RPA), and others (reviewed in, for example, Yan et al., 2014, Mol. BioSyst., 10:970-1003).

Alternatively, probe amplification methods include ligase chain reaction (LCR), Invader assay, Padlock probes, rolling circle amplification (RCA), and detection via the self-assembly of DNA probes to give supramolecular structures. Signal amplification strategies, as with probe amplification methods, do not require target nucleic acid amplification, and include Nicking endonuclease signal amplification (NESA) and nicking endonuclease assisted nanoparticle activation (NENNA), Junction or Y-probes, split DNAZyme and deoxyribozyme amplification strategies, template-directed chemical reactions that lead to amplified signals, non-covalent DNA catalytic reactions, hybridization chain reactions (HCR), Tyramide Signal Amplification, and Branched DNA (bDNA) (reviewed in Andras et al., 2001, Mol. Biotech., 19(1):29-44; and Yan et al., 2014, Mol. BioSyst., 10:970-1003).

With the progress of nanotechnologies, signal detection systems quickly adopted the use of bioconjugated nanoparticles (NPs) and a variety of detection systems were developed (reviewed, for example, in Ju et al., 2011, Chapter 2 in NanoBiosensing, pp 39-84). Most of the nucleic acid detection methods employ optical, visual, and/or electrochemical signal detection systems. However, with the advances in nanomaterial and nanofabrication technologies, there are new opportunities for miniaturization, further increasing sensitivity, and read-out digitalization for simple and cost effective point-of-use devices with integrated electronic chips/sensors. To adapt to these new opportunities, new methods and detection technologies need to be developed. In the present disclosure, novel methods and systems of electronically detecting nucleic acids are disclosed.

The present disclosure describes methods and systems for electronically detecting nucleic acids in which many of the constraints of existing nucleic acid detection systems are relaxed, including complexity, cost, and sensitivity. The nanostructure-based detection methods and systems described herein can detect a nucleic acid target in a very short time with high sensitivity (e.g., single molecule detection).

Described herein are methods and systems for detecting nucleic acids using a nanopore. The methods can accurately detect individual detectable positively-charged tags, the presence of which are correlated with a cleavage event of the probe, such as upon probe hybridization to complementary target nucleic acids. These positively-charged tags then are passed through a nanopore and can be detected. In this way, the presence of the target nucleic acid can be detected. When multiple targets are interrogated simultaneously, different positively-charged tags (i.e., unique for each target) can be used and, upon release, identified as they pass through the nanopore, thus identifying the presence of each target nucleic acid. The release of the detectable positively-charged tags upon cleavage of the probe can be detected in real-time.

The methods described herein are single-molecule digital detection methods, because the nanopore can detect a single tag molecule passing through. Thus, the number of probe cleavage events and, hence, the number of target-probe complexes formed and, hence, the number of target molecules, can be detected and quantitated. When the target nucleic acid is amplified, the signal is generated by each of the clonal molecules, which allows the methods described herein to be used for real-time quantification of amplification.

FIG. 5A schematically illustrates a method for direct detection of a target nucleic acid without target amplification. The method comprises providing a sample containing target polynucleotide and hybridizing a probe to the target to form a duplex. Methods and compositions of hybridization are well known in the art and may comprise denaturation of the sample nucleic acid prior, or simultaneously with addition of the probe. Hybridized probe forms a complex with the target polynucleotide, wherein the detectable tag forms a “flap”. The detectable tag can be a 5′ flap and/or a 3′ flap.

A scissile linkage located within the complementary portion of the probe becomes amenable for enzymatic cleavage upon forming a duplex structure. In some embodiments, the scissile linkage is the abasic site (AP site), e.g. tetrahydrofuran (THF), which can be cleaved by a number of endodeoxyribonucleases or DNA AP-lyases. Exemplary commercially available enzymes include Endonuclease IV, Fpg, hOOG1, Endonuclease VIII,

Exonuclease III, Endonuclease III (Nth), and APE1. The thermostable homologs of some of these enzymes also are available, e.g. Tma Endonuclease III and Tth Endonuclease IV. In some embodiments, the scissile linkage is a single ribonucleotide residue, which is recognized in the context of duplex polynucleotide and cleaved by RNaseH II. In some embodiment, the scissile linkage is four purine rich ribonucleotide residues, which is recognized and cleaved by RNaseH I. In some embodiments, the scissile linkage is 8-oxoguanine, which is cleavable by Fpg N-glycosylase in combination with AP lyase.

In some embodiments (see FIG. 5B), a scissile linkage is not provided and the detectable positively-charged tag (e.g., a 5′ or 3′ flap) is cleaved off from the probe by the 5′-3′ exonuclease activity or the flap endonuclease activity of DNA polymerase, e.g. E. coli Polymerase I, Bst Polymerase, or Taq Polymerase. This can be accomplished using a single oligonucleotide primer annealed to the target nucleic acid upstream of the target-annealed portion of the probe, such that there is no gap or a 1 to 2 nt gap between the primer and the complementary moiety of the probe. The design of the primer and the probe is such that, upon addition of at least one, or two, or three types of deoxyribonucleotides (dNTPs), e.g. dATP alone, or dATP and dTTP, or dATP, dTTP, and dGTP, the polymerase advances downstream along the target nucleic acid and displaces one or several bases of the target-complementary moiety of the probe to generate a short, 5′ or 3′ flap, which is cleavable by the 5′-3′ exonuclease (or flap endonuclease) activity of the DNA polymerase. These conditions prevent bulk synthesis of double stranded DNA by the polymerase because of the absence of all four nucleotides in the reaction mix, but still results in the cleavage of the probe to generate the detectable tag.

As a result of cleavage by endodeoxyribonuclease or DNA AP-lyase (see FIG. 5A), a nick or 1 nucleotide gap is formed in the probe, which releases the detectable positively-charged tag upon mild raising of temperature but may not necessarily release the rest of the annealed probe, as the rest of the annealed probe can be designed to have a more stable duplex structure with the target nucleic acid. In the method depicted in FIG. 5B, the detectable positively-charged tag can be released from the target polynucleotide without elevating the temperature. Even if the target complementary portion of the probe is partially degraded by the 5′-3′ exonuclease activity of the DNA polymerase and spontaneously released, the global charge of the released remnant is negative, so it will stay in the cis-chamber when an electrical potential is applied across the nanopore. In contrast, a properly cleaved positively-charged tag will have a global positive charge, which will flow through the nanopore (e.g., from the cis-chamber towards the negative electrode) and generate a change in conductance, which is registered as an electric signal having a certain amplitude and duration. Thus, every released positively-charged flap will be detected individually, and the total number of signals will correspond to the number of nucleic acid targets in the sample.

FIG. 6A schematically illustrates an exemplary method for amplification-based electronic detection of target nucleic acids using a nanopore detector. The method includes providing a sample containing target nucleic acid, oligonucleotide primers, and at least one probe. The target nucleic acid is amplified using forward and reverse primers and is detected using a probe. In some embodiments, the amplification method utilizes polymerase chain reaction (PCR). In some embodiments, the amplification method utilizes isothermal amplification method, e.g. recombinase polymerase amplification (RPA). The use of an isothermal amplification method greatly simplifies the construction of the microfluidic device as described herein, yet provides a fast, exponential amplification of the target nucleic acid sequence. During amplification, the probe anneals to the target amplicon region to form a double-stranded complex and is cleaved at a scissile linkage to release the detectable positively-charged tag. As indicated herein, the detectable positively-charged tag can be in the form of a 5′ and/or a 3′ flap. A scissile linkage located within the complementary portion of the probe becomes amenable for enzymatic cleavage only upon formation of a duplex structure. The released tag is subjected to an electrical field force and is translocated through the nanopore to generate an electrical signal, e.g. a change in conductance. The other nucleic acid species (i.e., the non-target nucleic acids) are negatively charged and remain in the cis-compartment. In some embodiments, the scissile linkage is an abasic site (AP site), e.g. tetrahydrofuran (THF), which can be cleaved by a number of endodeoxyribonucleases or DNA AP-lyases. Most of these enzymes are active at physiological temperatures and isothermal amplification techniques at such temperatures must be used. Exemplary methods include, e.g., recombinase polymerase amplification (RPA), which can take place at temperatures between 25° C. and 42° C.

The endodeoxyribonucleases and/or DNA AP-lyases can be selected from a list of enzymes that do not generate a 3′ hydroxyl group at the site of cleavage, as 3′ hydroxyl groups can be extended by the DNA polymerase, which would produce non-productive truncated secondary amplicons. Examples of such enzymes include Endo III and hOOG1, which produce a 3′-phospho-alpha, beta-unsaturated aldehyde at the 3′ end of the created termini. Additional examples of such enzymes include Endo VIII, Fpg, and hNEIL1, all of which create non-extendable 3′ phosphate termini. Further, RNA residues, which can be incorporated into the probe and are cleavable by Type I and Type II Ribonucleases H (RNase HI and RNase HII), are extendable scissile linkages since a 3′-OH group is generated at the 3′ end of the cleavage site. When several, e.g., three or four, RNA residues are used as a scissile linkage, which is cleaved by RNaseHI, the 3′-OH group resides preferentially on the second RNA residue. When a single RNA residue is used as a scissile linkage and cleaved by RNaseHII, a 3′-OH group on the DNA is produced. In both scenarios, the 3′-OH ends on either the DNA or RNA can serve as a template for polymerase-driven 3′ end extension by such mesophilic DNA polymerases as E. coli DNA polymerase I and Bsu DNA polymerase.

Thermostability of an enzyme permits it to be used at temperatures that allow for the highest hybridization stringency, maximizing sensitivity and selectivity for specific DNA:RNA heteroduplexes while minimizing background due to nonspecific hybridization. When the amplification method involves high temperature cycling, such as in PCR, a suitable scissile linkage in the probe typically includes at least four RNA residues. In some embodiments, a scissile linkage includes four purine-containing RNA residues, which is the substrate for Ribonuclease HI (RNase HI). RNase HI is an endoribonuclease that specifically hydrolyzes the phosphodiester bonds of an RNA tract that is annealed to complementary DNA. The resultant nick is located within the RNA tract and the 3′-OH group is located predominantly at the second RNA residue. Such an RNA-containing 3′-end cannot serve as a substrate for Taq polymerase. Thermostable RNase HI is commercially available as Hybridase (Epicentre), and specifically degrades the RNA in a DNA:RNA hybrid without affecting the DNA or unhybridized RNA. In contrast to E. coli RNase H, which is rapidly inactivated at 55° C., Hybridase RNase H is active at high temperatures (e.g., it has optimal activity above 65° C. and can be used at temperatures up to 95° C.). Thermostable RNase H type 2 enzyme is commercially available as Pyrococcus abysii RNase H2 (IDT). RNase HII is an endoribonuclease that recognize a single RNA residue within double-stranded DNA and preferentially nicks 5′ to a ribonucleotide. This produces a 3′-OH group on DNA residue, which can be extendable by any polymerase, including Taq polymerase, and, thus, is not suitable for the method of present disclosure. When the target amplification and detection method shown in FIG. 6A is carried out isothermally but at higher than physiological temperatures, the thermostable AP endonucleases and lyases can be used to cleave the scissile linkage, e.g. Pyrobaculum aerophilum Endonuclease III, and Thermotoga maritima Endonuclease IV.

FIG. 6B shows another exemplary method for amplification-based electronic detection of target nucleic acid using a nanopore. In some embodiments, the methods described herein includes providing a sample containing target polynucleotide, oligonucleotide primers, and a probe, and the target DNA is amplified using forward and reverse primers and a probe. In some embodiments, the amplification method can utilize polymerase chain reaction (PCR). The probe used for this method typically is complementary to the target sequence amplicon and does not overlap with the amplification primers used. The probe also usually does not contain a scissile linkage. Upon hybridization of such a probe to the target nucleic acid, the positively charged detectable tag is a 5′-flap, as depicted in FIG. 6B. DNA polymerase with 5′-3′ exonuclease activity and flap structure-specific endonuclease activity catalyzes hydrolytic cleavage of the phosphodiester bond at the junction of the single- and double-stranded DNA formed after the polymerase reaches the annealed probe and displaces one or more nucleotides of the target complementary portion of the probe. After cleavage of the probe, the flap structure (i.e., the detectable positively-charged tag) and a few negatively-charged DNA residues are released from the target nucleic acid. After the 5′-flap cleavage, the 5′-3′ exonuclease activity of the DNA polymerase digests the rest of the annealed target complementary portion of the probe. The released tag with the net positive charge is subjected to an electrical field force, which causes it to flow through the nanopore and generate an electrical signal, e.g., a change in conductance. Other nucleic acid species of the amplification reaction (primers, probes, target nucleic acids, as well as amplification and 5′-3′ exonuclease degradation products) are negatively charged and remain in the cis-compartment. Exemplary DNA polymerases with 5′-3′ exonuclease activity include Taq DNA polymerase, Bst DNA polymerase, and E. coli DNA polymerase I. The latter is not a thermostable enzyme and can't be used in thermocycling conditions required in standard PCR reactions.

In some embodiments, the detectable tag is at the 3′ end of the target complementary portion of the probe. FIG. 6C shows an exemplary method for amplification-based electronic detection of target nucleic acid using a nanopore, where the detectable tag is shown as a 3′ flap. In some embodiments, the method includes providing a sample containing target polynucleotide, oligonucleotide primers, and a probe. The target DNA can be amplified using forward and reverse primers in the presence of a probe. In some embodiments, the amplification method can be the polymerase chain reaction (PCR). The probe used in this method typically is complementary to the target sequence amplicon but does not overlap with the amplification primers used. The probe also does not necessarily contain a scissile linkage. Upon hybridization of such a probe to the target nucleic acid, the positively charged detectable tag is a 3′-flap (FIG. 6C). When DNA polymerase with its 5′-3′ exonuclease activity reaches the annealed probe, it continues to copy the template via nick-translation, where the probe is degraded/digested from the 5′-end until the Tₘ of the remaining undegraded complementary portion drops down to the point where it dissociates from the target sequence together with the detectable tag. In this embodiment, the released detectable tag comprises the 3′ end nucleotides of the target complementary portion of the probe (negatively charged) and a positively charged 3′ flap.

The net charge of such a detectable tag depends on the number of negatively charged nucleotides and the number of positive charges of the 3′ flap. The lesser the number of undigested nucleotides in the released tag, the higher the net positive charge. The positively charged 3′-flap of the probe can stabilize the 3′ portion of the complementary moiety of the probe by electrostatic interactions with the template strand, thereby decreasing the number of nucleotides in the released tag. In some embodiments, the portion of the target-complementary moiety of the probe adjacent to the junction with the flap can include one or more duplex-stabilizing nucleotide analogs with negative (e.g. LNA), or neutral (e.g. PNA, PMO, methylphosphonate) or positive (e.g. PMOplus) backbones. The use of such modifications in the backbone decreases the number of negatively charged nucleotides in the detectable tag or replaces them with residues having neutral or positive charge. Moreover, these modified backbones also exhibit an increased resistance to degradation by nucleases (e.g., by the 5′ exonuclease of DNA polymerase). In some embodiments, a nucleotide analog having a neutral or a cationic backbone can be PMO, PMOplus, PNA, methylphosphonates, or any combination thereof. The released tag with the net positive charge then can be subjected to an electrical field force such that it flows through the nanopore and generates an electrical signal, e.g. a change in conductance. Other nucleic acid species of the amplification reaction (e.g., primers, probes, target nucleic acids, as well as amplification and 5′-3′ exonuclease degradation products) are negatively charged and remain in the cis-compartment.

The primers and probes used in the methods described herein can have any of a variety of lengths and configurations suitable for efficient target hybridization and amplification and suitable for producing a detectable tag. Typically, primers and probes can be from about 15 to about 30 nucleotides in length (e.g., from about 20 to about 25 nucleotides), although lengths outside of these ranges also may be used. Shorter lengths can be used when a primer contains one or more nucleotide analogs having enhanced base pairing affinities such as, for example, locked nucleic acids (LNAs) or peptide nucleic acids (PNAs). The first and second primers can be designed to anneal to the target nucleic acid so as to produce an amplified product of any desired length, usually at least 30 (e.g., at least 50) nucleotides in length and up to 200 (e.g., up to 300, 500, 1000) or more nucleotides in length. The probes and primers may be provided at any suitable concentrations.

### 3. Nucleic Acids

Target nucleic acids used in the electronic detection and identification methods and systems described herein may be single-stranded or double-stranded, or may contain portions of double-stranded and single-stranded nucleic acids. For example, target nucleic acids can be genomic DNA, mitochondrial DNA, cDNA, mRNA, ribosomal RNA, small RNA, non-coding RNA, small nuclear RNA, small nucleolar RNA, and Y RNA. In some embodiments, the target nucleic acids can be extracted and/or purified from a sample.

Target nucleic acids (e.g., genomic DNA) can be obtained from any organism of interest. Organisms of interest include, for example, animals (e.g., mammals, including humans and non-human primates); plants, fungi, and pathogens such as bacteria and viruses. In some embodiments, the target nucleic acids (e.g., genomic DNA or RNA) are bacterial or viral nucleic acids.

Target nucleic acids can be obtained from samples of interest. Non-limiting examples of samples include cells; bodily fluids (including, but not limited to, blood, urine, serum, lymph, saliva, anal and vaginal secretions, perspiration and semen); environmental samples (for example, air, agricultural, water and soil samples); biological warfare agent samples; research samples (e.g., products of nucleic acid amplification reactions, such as PCR or MDA amplification reactions); purified samples, such as purified genomic DNA; RNA preparations; and raw samples (bacteria, virus, genomic DNA, etc.). Methods of obtaining target nucleic acids (e.g., genomic DNA) from organisms are well known in the art. See, e.g., Sambrook et al., Molecular Cloning: A Laboratory Manual (1999); Ausubel et al., eds., Current Protocols in Molecular Biology, (John Wiley and Sons, Inc., NY, 1999), or the like.

In some embodiments, target nucleic acids are genomic DNA. In some embodiments, target nucleic acids are a subset of a genome (e.g., a subset of interest for a particular application, e.g., selected genes that may harbor mutations in a particular subset of a population such as patients with cancer). In some embodiments, target nucleic acids are exome DNA, i.e., a subset of whole genomic DNA enriched for transcribed sequences. In some embodiments, target nucleic acids are all or part of a transcriptome, i.e., the set of all mRNA or “transcripts” produced in a cell or population of cells.

In some embodiments, target nucleic acids (e.g., genomic DNA) are fragmented before using. Any method of fragmentation can be used. For example, in some embodiments, the target nucleic acids are fragmented by mechanical means (e.g., ultrasonic shearing, acoustic shearing, or needle shearing); by chemical methods; or by enzymatic methods (e.g., using endonucleases). Methods of fragmentation are known in the art (see, e.g., US 2012/0004126). In some embodiments, fragmentation can be accomplished using ultrasound.

In some embodiment, the methods described herein include isolating the target nucleic acids from a sample and preparing the target nucleic acids for electronic detection. Some exemplary techniques for extracting nucleic acids from samples of various origins include using lysing enzymes, ultra-sonication, high pressure, or any combination thereof. In many cases, upon release from the cell, nucleic acids can be purified from cell wall debris, proteins and other components by commercially available methods including, for example, use of proteinases, organic solvents, desalting, spin columns, and binding to functionalized matrices including magnetic nanoparticles. In some cases, the target nucleic acid is cell-free nucleic acid (e.g. liquid biopsies) and does not require extraction from a cell.

4. Probes

Typically, a probe used in the methods described herein includes two parts: a target-complementary oligonucleotide 100 and a positively-charged detectable tag 101 (see, for example, FIG. 1). The target complementary oligonucleotide has a phosphodiester backbone such that it has a negative charge in aqueous solutions of neutral pH. A phosphodiester backbone generally includes a sugar-phosphate backbone of alternating sugar and phosphate moieties, with a nucleotide base (generally, a purine or a pyrimidine group) attached to each sugar moiety. Any sugar(s) such as ribose (for RNA), deoxyribose (for DNA), arabinose, hexose, 2′-fluororibose, and/or a structural analog of a sugar, among others, can be included in the backbone. In some embodiments, one or more residues in the complementary oligonucleotide can be substituted with nucleotide analogs of alternative backbones. Exemplary alternative backbones can include: a) negatively charged phosphoramidates (see, e.g., Beaucage et al., 1993, Tetrahedron, 49(10):1925), phosphorothioates (PS) (see, e.g., Mag et al., 1991, Nucleic Acids Res., 19:1437; and U.S. Pat. No. 5,644,048), locked nucleic acids (LNA) (see, e.g., Koshkin et al., 1998, Tetrahedron, 54:3607-30; WO 98/39352; WO 99/14226; WO 00/56746; and WO 99/60855;), unlocked nucleic acids (UNA) (see, e.g., Pasternak et al., 2011, Org. Biomol. Chem., 9:3591-97), N3′-P5′ phosphoroamidates, 2′-O-methoxyethyl (2′-MOE) RNA, 2′-O-methyl (2′-OMe) RNA (see, e.g., Kole et al., 2012, Nat. Rev. Drug Discov., 11(2):125-40), hexitol nucleic acid (HNA); b) uncharged (neutral) methylphosphonates (see, e.g., Miller et al., 1981, Biochemistry, 20:1874-80), phosphorodiamidate morpholino oligomers (PMOs) (see, e.g., U.S. Pat. No. 5,185,444), peptide nucleic acid (PNA) (see, e.g., Egholm, 1992, J. Am. Chem. Soc., 114:1895; Braasch and Corey, 2001, Chem. Biol., 8(1):1-7; Nielsen, 1995, Annu. Rev. Biophys. Biomol. Struct., 24:167-83; Nielsen et al., 1999, Curr. Issues Mol. Biol., 1(1-2):89-104; and Ray et al., 2000, FASEB J., 14(9):1041-60), triazole-linked DNA (see, e.g., Varizhuk et al., 2013, J. Org. Chem., 78(12):5964-69). Nucleic acids with artificial backbones and/or moieties can be used to increase or reduce the total charge, increase or reduce base-pairing stability, increase or reduce chemical stability, to alter the ability to be acted on by a reagent, and the like.

In some embodiments, the entire length of the target complementary moiety of probe is from about 15 to about 60 bases in length (e.g., about 15, about 20, about 25, about 30, about 35, about 40, about 45, about 50, about 55, or about 60 bases in length).

In some embodiments, a probe includes 3′, or 5′, or both 3′ and 5′ termini blocked for extension by polymerase and /or degradation by nucleases. To block 3′ end extension by polymerase, the terminal nucleotide can be modified to remove or substitute 3′-OH group with a blocking group (e.g., alkyl groups, non-nucleotide linkers, alkane-diol, dideoxynucleotide residues, and cordycepin). Non-limiting examples of commercially available 3′ modifications or blocking groups include, for example, a 3′ amino modifier (3AmMO, Integrated DNA Technologies (IDT), Coralville, Iowa), 3′ spacer (e.g., C3 spacer 3SpC3, Integrated DNA Technologies (IDT)), a dideoxynucleotide (e.g. ddC, Integrated DNA Technologies (IDT)), an inverted dT (invdT, Integrated DNA Technologies (IDT)), or 3-dT-Q/3-dA-Q/3-dC-Q/3-dG-Q (Operon/Eurofins, Huntsville, Ala.). 5′ and/or 3′ termini can be blocked from exonuclease degradation by a modified 5′ or 3′ linkage (see, e.g., WO 93/13121), modified nucleotides, and non-nucleotide exonuclease resistant structures. Exemplary end-blocking groups include cap structures (e.g., a 7-methylguanosine cap), inverted nucleomonomers (e.g., with 3′-3′ and/or 5′-5′ end inversions, see, e.g., Ortiagao et al., 1992, Antisense Res. Dev., 2:129), methylphosphonate, phosphoramidite, non-nucleotide groups (e.g., non-nucleotide linkers, amino linkers, conjugates) and the like. To reduce nuclease degradation, at least one 5′ and /or 3′most linkage can be a modified linkage, e.g., a phosphorothioate linkage.

In some embodiments, the probe comprises a scissile linkage 102 within the target complementary moiety 100 (see, for example, FIG. 1B and FIG. 1D). Non-limiting examples of scissile linkages include at least one RNA residue; an oxidized purine; an oxidized pyrimidine; an apurinic, or apyrimidinic (AP) site; any of deoxyuridine, 5-hyroxyuracil, 5-hydroxymethyluracil, or 5-formyluracil; or an abasic nucleotide analog, tetrahydrofuran (THF—sometimes referred to as a ‘dSpacer’). In some embodiments, the scissile linkage is cleaved only when the probe forms a double-stranded nucleic acid complex with the single-stranded target nucleic acid within the region of complementarity.

Exemplary oxidized purine, 8-oxo-guanine, which can be incorporated into oligonucleotide during syntheses, can be cleaved by the Fpg (formamidopyrimidine [fapy]-DNA glycosylase, also known as 8-oxoguanine DNA glycosylase), when paired with deoxy-cytosine or deoxy-guanosine within double-strand DNA. The enzyme acts both as N-glycosylase and an AP-lyase. The N-glycosylase activity releases a damaged purine, 8-oxoguanine, from double stranded DNA, generating an apurinic (AP site). The AP-lyase activity, then, cleaves both 3′ and 5′ to the AP site, thereby removing the AP site and leaving a 1 base gap. A similar enzyme, hOGG1 (the alpha isoform), is an 8-oxoguanine DNA glycosylase that acts both as an N-glycosylase and an AP-lyase. In contrast to Fpg, the AP-lyase activity of hOOG1 cleaves only 3′ to the AP site, leaving a nick with 5′ phosphate and a 3′-phospho-alpha, beta-unsaturated aldehyde.

Apurinic or apyrimidinic (AP) sites, generated by N-glycosylases, or integrated into the probe as tetrahydrofuran (THF, or dSpacer), is cleaved by AP nucleases (e.g., endodeoxyribonucleases) and DNA AP-lyases (e.g., Endo IV endonuclease, AP lyase, FPG glycosylase/AP lyase, Endo VIII glycosylase/AP lyase, Exonuclease III (E. coli)). Endo IV cleaves at the first phosphodiester bond that is 5′ to the lesion, leaving a hydroxyl group at the 3′ terminus and a deoxyribose 5′-phosphate at the 5′ terminus. Tth Endonuclease IV is a thermostable homologue of Endo IV. Endonuclease VIII (E. coli) acts as both an N-glycosylase and an AP-lyase. The AP-lyase activity cleaves 3′ and 5′ to the AP site, leaving a 5′ phosphate and a 3′ phosphate. While Endonuclease VIII is similar to Endonuclease III, Endonuclease VIII has beta and delta lyase activity, while Endonuclease III has beta lyase activity. Endo VIII also has intrinsic N-glycosylase activity, which releases damaged pyrimidines (e.g., urea, 5, 6-dihydroxythymine, thymine glycol, 5-hydroxy-5-methylhydanton, uracil glycol, 6-hydroxy-5, 6-dihydrothymine and methyltartronylurea) from double-stranded DNA, generating an apurinic (AP site). Endonuclease III (Nth) protein from Escherichia coli also acts as both an N-glycosylase and an AP-lyase. The AP-lyase activity of the enzyme cleaves 3′ to the AP site, leaving a 5′ phosphate and a 3′ ring opened sugar. Endo III also has N-glycosylase activity, which releases damaged pyrimidines (e.g., urea, 5, 6 dihydroxythymine, thymine glycol, 5-hydroxy-5 methylhydanton, uracil glycol, 6-hydroxy-5, 6-dihdrothimine and methyltartronylurea) from double-stranded DNA, generating a basic (AP site). The thermostable homolog of the E. coli Endonuclease III (Nth) is known as Tma Endonuclease III. The E. coli exonuclease III (AP endonuclease VI) is a DNA-repair enzyme that hydrolyzes the phosphodiester bond 5′ to an abasic site in both double-stranded and single-stranded DNA (see, e.g., Shida et al., 1996, Nucleic Acids Res., 24(22):4572-76). Human apurinic/apyrimidinic (AP) endonuclease, APE 1, shares homology with E. coli exonuclease III protein. APE 1 cleaves the phosphodiester backbone immediately 5′ to an AP site via a hydrolytic mechanism to generate a single-strand DNA break, leaving a 3′-hydroxyl and 5′-deoxyribose phosphate terminus.

Deoxyuridine within a probe is recognized and released by the N-glycosylase activity of uracil DNA glycosylase (UDG), generating the AP site. The AP site then is cleaved by AP nucleases (endodeoxyribonucleases) or DNA AP-lyases (e.g., Endo IV endonuclease, AP lyase, FPG glycosylase/AP lyase, Endo VIII glycosylase/AP lyase. Deoxyuridine can be cleaved when paired with deoxy-adenosine in the double-stranded probe-target complex, but also within the single-stranded probe, which requires inactivation of UDG by Uracil DNA inhibitor (New England Biolabs, Mass.) or the removal of the probe before cleavage of the AP site. Abasic sites also can be generated at nucleotide analogues other than deoxyuridine and cleaved in an analogous manner by treatment with endonuclease. For example, deoxyinosine can be converted to an abasic site by exposure to AlkA glycosylase. The abasic sites thus generated then can be cleaved, typically by treatment with a suitable endonuclease (e.g. Endo IV, AP lyase). See, for example, US 2011/0014657).

For simultaneous detection of multiple polynucleotide targets (multiplexed detection), a plurality of different probes can be used. The plurality of probes can include different detectable tags 101 (see FIG. 1). In one example, the probes have tags 201, 202, and 203 with variable number of charges as exemplified in FIG. 2A. The example of the electrical signal generated by such multiplexed probes is shown is FIG. 2B. The identity of the tag can be established by the uniqueness of the electrical signal generated. Correspondingly, the identified tags can be correlated with the individual target nucleic acid present in the sample. In another example the detectable tags 305, 306, and 307 (see FIG. 3A) include positively charged moieties 301 adjacent to the target complementary moiety 300 and additional chemical compounds (or chemical groups) 302, 303, and 304 attached to the moiety 301. These compounds can have a neutral charge, or can add additional positive charge(s) to moiety 301, but they are intended to differentiate the detectable tags 305, 306, and 307 by shape, size, and charge distribution, or any combination thereof. An example of the electrical signal generated by detectable tags of this type of multiplexed probes is shown is FIG. 3C. In another example of the use of multiplexed probes, the positively charged moieties 301 of the detectable tags 308, 309, and 310 (see FIG. 3B) are separated from the target complementary moiety 300 portion of the probe by the chemical groups (or chemical compounds) 302, 303, and 304 to prevent potential negative interference caused by the proximity of the positively charged moiety 301 on the efficiency of cleavage of the scissile linkage 102. In an additional example, the probe can include two detectable tags 401 and 403 (see FIG. 4A), 403 and 406 (see FIG. 4B), or 406 and 407 (see FIG. 4C) attached to the 5′ and 3′ ends of the target complementary moiety 400 of the probe. The probes with two detectable tags can include two scissile linkages 401 and 402, which can be cleaved enzymatically to release both tags. Not all combinations of two detectable tags are shown, but all combinations are included in this disclosure. The detectable tags 201, 202, and 203 (see FIGS. 2), 305, 306, and 307 (see FIG. 3A), and 308, 309, and 310 (see FIG. 3B) are shown as a 5′ flaps only for the purpose of providing an example—these probes can be attached to the 3′ end of the target complementary moiety of the probe as 3′ flaps.

### 5. Exemplary Tags

In some embodiments, the positively charged detectable tag 101 (e.g. see FIG. 1) can be a polynucleotide, or polynucleotide analog with a positively charged backbone. Exemplary suitable positively charged backbones useful in the subject methods include deoxynucleic guanidine (DNG) (see, e.g., Dempcy et al., 1995, Proc. Natl. Acad. Sci. USA, 92:6097-101; Barawkar and Bruice, 1998, Proc. Natl. Acad. Sci. USA, 95:11047-52; Park and Bruice, 2008, Bioorg. Med. Chem. Lett., 18(12):3488-91), a nucleosyl amino acid (NAA)-modification (see, e.g., Schmidtgall et al., 2015, Beilstein J. Org. Chem., 11:50-60), N, N-diethyl-ethylenediamine phosphoramidate linkages (see, e.g., Dagle and Weeks, 1996, Nucl. Acids Res., 24(11):2143-49), triazole-linked Plus DNA (TL DNA+) (see, e.g., Fujino et al., 2013, Heterocycles, 87(5):1023-28), or positively charged piperazine residues in a backbone of phosphorodiamidate morpholino PMOs (PMOplus). In some embodiments, a positively charged tag 101 can be a chimera of a polynucleotide (or polynucleotide analog) and a peptide. Exemplary chimeras can include a positively-charged arginine-rich peptides in peptide-conjugated phosphorodiamidate morpholino PMOs (PPMOs) and peptide nucleic acids (PNA) oligomers bearing positively charged lysine (Lys) tails. Conjugates between a peptide and nucleic acid can be prepared using techniques generally known in the art. In one such technique, a peptide and nucleic acid components of the desired amino acid and nucleotide sequence can be synthesized separately, e.g., by standard automated chemical synthesis techniques, and then conjugated in an aqueous/organic solution. By way of example, the OPeC™ system commercially available from Glen Research, is based on the native ligation of an N-terminal thioester-functionalized peptide to a 5′-cysteinyl oligonucleotide.

In some embodiments, a positively charged detectable tag 101 (see, e.g., FIG. 1) includes an oligocation or oligonucleotide-oligocation conjugate. Exemplary positively charged conjugates include Zip nucleic acids (ZNAs) (see, e.g., Moreau et al., 2009, Nucleic Acids Res., 37(19):e130; Paris et al., 2010, Nucleic Acids Res., 38(7):e95). The global charge of ZNA is modulated by the number of cationic spermine moieties attached to the oligonucleotide. In some embodiments, a probe can be synthetized as ZNA to include the target complementary moiety 100, represented by the oligonucleotide or oligonucleotide analog, and a tag 101 of spermine units. In some embodiments, upon cleavage of the tag by the 5′ nuclease activity of polymerase, or at a scissile linkage 102 by enzymatic activities as described above, the released detectable tag 101 can include positively charged polyspermine and at least one negatively charged nucleotide residue. According to the methods described herein, the number of cationic spermine units should be selected such that the global (or net) charge of the released detectable tag is positive, while the global (or net) charge of the probe is negative.

In some embodiments, the number of positively charged nucleotides, or their analogs, or non-polynucleotide cations constituting the positively charged detectable tags 201, 202, and 203 can be different in different probes (e.g., different probes used for multiplexed detection of polynucleotide targets as depicted in FIG. 2A).

In some embodiments, an additional chemical group or compound (e.g., 302, 303, and 304 of detectable tags 305, 306, 307, 308, 309, and 310 shown in FIG. 3) attached to the positively charged moieties of the detectable tags used for multiplexing includes a polyethylene glycol (PEG) chain. This approach is exemplified by the attachment of twelve ethylene glycol units to oligonucleotides with phosphodiester and phosphorothioate backbones (see, e.g., Shokrzadeh et al., 2014, Bioorg. Med. Chem. Lett., 24(24):5758-61).

In some embodiments, an additional chemical group or compound can include positively-charged glycopolymers. Methods of producing methacrylate or methacrylamide-based sugar monomers and polymerizing them by RAFT or ATRP to yield linear or branched glycopolymers with narrow polydispersity are well known. Also, oligosaccharide-based monomers can be polymerized to obtain well-defined glycopolymers. These glycopolymers can be conjugated to oligo- and polynucleotides using various methods, e.g., via disulfide bond exchange (see, e.g., Engineered Carbohydrate-Based Materials for Biomedical Applications, Ed. Ravin Narain, 2001, Ch 4, Willey). Oligonucleotide-glycopolymer conjugates bearing alpha-mannosides and beta-galactosides can be prepared by coupling 5′-thiol-modified oligonucleotides with iodoacetamidated glycopolymers that were synthesized by telomerization (see, e.g., Akasaka et al., 2001, Bioconjugate Chem., 12(5):776-85). These conjugates minimally affect the DNA conformation and melting behavior of the duplex.

In some embodiments, the additional uncharged or positively charged chemical group or compound includes polypeptides and/or polypeptoids (i.e., poly-N-substituted glycines). In some embodiments, the chemical moieties are a derivative thereof, such as N-methoxyethylglycine (NMEG) oligomers (see, e.g., U.S. Pat. No. 7,371,533; Meagher et al., 2008, Anal. Chem., 80:2842-48).

In some embodiments, additional uncharged or positively-charged chemical groups or compounds can include various commercially available oligonucleotide spacers or arms. Non-limiting examples of spacers and arms include 3′ hexandiol (a six carbon glycol spacer), Spacer 9 (triethylene glycol spacer that can be incorporated at the 5′-end or 3′-end of an oligo including consecutively whenever a longer spacer is required), Spacer 18 (an 18-atom hexa-ethyleneglycol spacer, which can be incorporated at the 5′ end or at the 3′ end). In addition, Spacer C12 (a 12-carbon spacer that is used to incorporate a long spacer arm into an oligonucleotide) can be incorporated in consecutive additions if a longer spacer is required (Tri Link Inc.). In another example, spacer phosphoramidites (Fidelity Systems Inc.) are available in different lengths and variable hydrophobicity and can be added alone or sequentially to the 5′-end of an oligonucleotide during synthesis (e.g., Arm26-Ach Spacer, Arm26-T Spacer, 15A Spacer, 14A Spacer, Diol 22A Spacer). These non-nucleosidic spacers of different lengths and variable hydrophobicity can be added alone or sequentially to the 5′-end and are made from a common secondary aminoalcohol, trans-4-aminocyclohexanol, which is subsequently derivatized utilizing proprietary MOX chemistry from Fidelity Systems Inc. Another way to vary the 5′ end modifications is to use Branching Unit 11 Amidite, which introduces a junction point at the 5′-end of an oligonucleotide. The branching units can be added alone or sequentially to make highly branched oligonucleotides.

### 6. Detection of Positively-Charged Tags

A target nucleic acid can be detected electronically in a sample by detecting the by-product of the cleavage of the probe, a positively-charged detectable tag. In some embodiments, a method for detecting a target nucleic acid molecule includes (a) cleaving the probe upon hybridization to the target polynucleotide, where, upon cleavage, positively-charged tags are produced, and (b) detecting the released positively-charged tag using a nanopore. The tag can be directed to flow through the nanopore by a voltage difference across the nanopore as described herein.

As a positively-charged tag passes through a nanopore, it will generate an electronic change. In some embodiments, the electronic change is a change in current amplitude, a change in conductance of the nanopore, or any combination thereof.

With continued reference to FIGS. 2, 3, and 4, a different tag can be attached to a target-complementary moiety of different target-specific probes such that, when the tags are released and pass through the nanopore, they can be differentiated from each other based on the particular signal that is generated in the nanopore. With particular reference to FIG. 3C, three different signal intensities (305, 306 and 307) can be detected. For example, one tag passing through the nanopore can generate a signal with an amplitude 305, another tag 306 can generate a signal with a lower amplitude, and the third tag 307 can generate a signal with a higher amplitude. In some cases, the signal may return to a baseline level 311 between detections. In some embodiments, the time during which a nanopore is blocked by a tag can be different between tags, where tags having a positively-charged portion and chemical compounds of different mass (“drags”), e.g., linear polymers of varying number of monomers, e.g., PEG. Translocation of such tags through a nanopore causes distinct mass-dependent conductance states with characteristic mean residence times. For example, Robertson et al. (2007, PNAS USA, 104(20):8207-11) demonstrated that the conductance-based mass spectrum clearly resolves the repeat units of ethylene glycol, and the residence time increases with the mass of the PEG.

The present disclosure provides methods for determining the identity of a positively-charged tag having an additional chemical compound (“drag”) (e.g., FIG. 3), which includes contacting the compound with a system that measures the conductance and records a change in the electric field when the detectable tag bearing the chemical compound translocates through the nanopore. A change in the electric field is the result of interaction between the tag and the compound, the electrolyte, and the pore, which is indicative of the size, charge, and composition of the compound, thereby allowing a correlation to be made between the change in the electrical field and the identity of the compound. In some cases, the detectable tags do not carry additional “drags” (FIG. 2), and are different only by the number of positively charged units of polymer constituting the tag.

In some embodiments, a method for determining the identity of a tag includes a conductance measurement system. A conductance measurement system can include a first and a second chamber having a first and a second electrolyte solution separated by a physical barrier. In such a system, the barrier has at least one nanopore having a diameter on a nanometer scale; means for applying an electric field across the barrier; and means for measuring change in the electric field.

In some embodiments, an electronic method for target nucleic acid detection includes a field-effect transistor (FET) to sense the positively charged tag. In some embodiments, the FET includes an ion-sensitive FET (ISFET) or a carbon-nanotube FET (CNFET).

### 7. Device

The methods and materials disclosed herein may be used in conjunction with any of a variety of apparatuses or devices. The electronic detection of the target nucleic acid disclosed herein can be advantageous for performing the disclosed methods in miniaturized format in conjunction with a microfluidic device. Microfluidic devices can utilize a variety of microchannels, wells, and/or valves located in various geometries in order to prepare, transport, and/or analyze samples. Fluids can be transported through the device using various forces, including injection, pumping, applied suction, capillary action, osmotic action, electro-osmosis, and thermal expansion and contraction, among others.

In some embodiments, a microfluidic device can include one or more microfluidic chambers or channels fabricated in a suitable substrate, an inlet configured to receive a sample containing at least one target polynucleotide sequence; and, embedded into the microfluidic device, a nanopore detector chip connected with the inlet via channels and valves.

In some embodiments, a plurality of chambers or channels can be used for splitting or dividing a sample containing the target nucleic acid and performing the methods of present disclosure in a singleplex format, i.e. different targets are interrogated individually in each reaction chamber. In some embodiments, such architecture of the microfluidic device, when additionally configured with multiple inlet ports, can be used for interrogating multiple samples for single (singleplex detection) or multiple (multiplex detection) targets in one reaction chamber.

In some embodiments, a nanopore detector chip includes one or more cis-chambers having a first electrode and configured for subjecting the sample to temperature control. Typically, the cis-chamber is where cleavage of the detectable tag from the probe occurs. In some embodiments, a nanopore detector chip includes one or more trans-chambers having a second electrode, which is separated from the cis-chamber by a barrier that includes the embedded nanopore. Such a nanopore detector chip is suitable for detecting and identifying a tag by applying an electric potential to the electrodes and forcing detectable tags to translocate through the nanopore, thereby causing a change in conductance.

FIG. 7 shows an example of a nanopore detector chip (or a microfluidic device). A nanopore detector can include a cis-chamber (top) 600 containing a top electrode 603 and conductive solution; a trans-chamber (bottom) 601 embedded in a semiconductor substrate 608 and containing a bottom conductive electrode 604; and an electrically resistant barrier or membrane separating the two chambers 600 and 601 and having an embedded nanopore 602. A nanopore detector also can include electrical circuitry 607 for controlling electrical stimulation (e.g. voltage bias) and for processing the detected electrical signal embedded within substrate (e.g., a silicon substrate); a variable voltage source 609 included as a part of the electric circuit 607; and a temperature controlling element 606. The electrical circuitry also may include amplifiers, integrators, noise filters, feedback control logic, and various additional components. The temperature control element 606 may be a thermoelectric heating and/or cooling device (e.g. Peltier element). The cis-chamber typically contains the target nucleic acids, enzyme mix, probes, and primers (when applicable), in a buffered electrolyte solution.

In some embodiments, the electrical circuitry of the nanopore detector may be coupled to a computer processor, which may be coupled to memory.

In some embodiments, multiple nanopore detectors may form a nanopore array, where nanopores may be individually addressable.

In some embodiments, during the detection of the detectable positively-charged tags, the voltage bias is provided such that the top electrode embedded in the cis-chamber is the positive electrode and the bottom electrode embedded in the trans-chamber is the negative electrode. Such voltage bias forces the detectable positively-charged tags to flow from the cis-chamber through the nanopore to the trans-chamber. This is opposite of conventional nucleic acid detection and/or sequencing nanopore devices known in the art, in which the negatively-charged nucleic acids translocate through the nanopore in the direction of the positive electrode.

A nanopore used in the methods and systems described herein can be a solid state nanopore fabricated with non-conductive material, or a biological nanopore formed with proteins capable of self-assembly into structures forming a channel and able to embed in a lipid bilayer.

Several biological nanopores can be used to detect nucleic acids at the single molecule level (see, e.g., Feng et al., 2015, Nanopore-based Fourth-generation DNA Sequencing Technology, Genomics, Proteomics & Bioinformatics, 13(1):4-16). One of them is alpha-Hemolysin (alpha-HL) pore. The inner diameter of the alpha-HL channel and a single-stranded DNA (ssDNA) molecule are very close in size (diameter˜1.3 nm). An alpha-HL nanopore is able to discriminate single nucleotides using ionic current inside the nanopore. The limiting aperture of the nanopore allows linear single-stranded but not double-stranded nucleic acid molecules (diameter˜2.0 nm) to pass through. Another nanopore is Mycobacterium smegmatis porin A (MspA); the channel of the MspA octamer is 1 nm in diameter at the minimal point, which is relatively small and narrow compared to that of alpha-HL. Thus, it can improve the spatial resolution of ssDNA detection and sequencing. Both alpha-HL and MspA are very robust and channel remains active under extreme experimental conditions, such as varying the pH value from 2 to 12 and maintaining the temperature at 100° C. for 30 min. Bacteriophage Phi29 nanopore is another biological nanopore. The channel in the phi29 pore has the cross-sectional area of about 10 nm² (3.6 nm in diameter) at one of the ends, and it has been demonstrated that double-stranded DNA (dsDNA) can pass through. Compared to alpha-HL and MspA, the phi29 pore has a larger diameter, which allows for the measurement of larger molecules, such as dsDNA, dsDNA coupled to bulky groups, such as complexes of DNA or proteins.

Although biological nanopores have shown useful for ssDNA sequencing, such protein pores have a constant pore size, profile and lack of stability. They suffer from the fragility of traditional supported lipid membranes. To overcome these deficiencies, various synthetic solid-state nanopores have been fabricated using different materials and methods and applied to nucleic acid analysis. Solid-state nanopores have many advantages over their biological counterparts, such as chemical, thermal, and mechanical stability, and size adjustability. Various techniques are often used to fabricate nanopores in silicon nitride (Si3N4), silicon dioxide (SiO2), aluminum oxide (Al2O3), boron nitride (BN), graphene, polymer membranes, and hybrid materials. Methods of fabricating nanopores include the ion milling track-etch method, electron beam based decomposition sputtering, focused ion beam (FIB) techniques, the laser ablation method, electron-beam lithography, helium ion microscopy, and dielectric breakdown methods. Electrical and geometric properties of solid-state nanopores give them a distinct advantage over their biological counterparts.

A nanopore can be formed or fabricated or otherwise embedded in a non-conductive barrier or a membrane disposed adjacent to a sensing electrode of a sensing circuit, such as an integrated circuit. An integrated circuit may be an application specific integrated circuit (ASIC). In some embodiments, an integrated circuit is a field effect transistor or a complementary metal-oxide semiconductor (CMOS).

### 8. Kits/Articles of Manufacture

In another aspect, kits for practicing the methods described herein are provided.

In some embodiments, a kit includes a first oligonucleotide primer and a second oligonucleotide primer to be used as forward and reverse primers for nucleic acid amplification; and a probe having a detectable tag for detection of target nucleic acid as described herein.

In some embodiments, a kit includes oligonucleotide primers and probes for detecting two or more target nucleic acids (e.g., oligonucleotide primers and a probe for detecting a first target nucleic acid and oligonucleotide primers and a probe for detecting a second target nucleic acid) as described herein.

In some embodiments, a kit also can include one or more additional components related to nucleic acid detection as described herein. In some embodiments, a kit can further include one or more enzymes for carrying out one or more of the method steps described herein (e.g., an enzyme for use in DNA amplification, an enzyme for use in cleavage of the probe, or an enzyme to perform reverse transcription of RNA), and optionally can include other reagents for performing an enzymatic reactions and/or detection of a tag as described herein (e.g., buffers, nucleotides, additives, etc.).

In some embodiments, a kit comprises components (e.g., oligonucleotide primers, probes, enzymes, and other reaction components) in easy to use pre-mixed formulations.

### Part B—Methods of Detecting Target Nucleic Acids Using Rolling Circle Replication

### 1. Overview

Strategies for nucleic acid detection can be grouped into three categories: target amplification, probe amplification, and signal amplification. A number of methods have been developed for target amplification, with polymerase chain reaction (PCR) currently being the gold standard in various diagnostic applications. Quantitative PCR using fluorescent probes, e.g., TaqMan dual-labeled probes, Scorpion probes, Molecular Beacons, LightCycler probes, and intercalating dyes (e.g.SYBR Green), is the most common method in nucleic acid diagnostics. Quantitative PCR requires sophisticated and expensive equipment for performing thermal cycling and optical readout. To circumvent limitations in PCR usage, especially in the point-of-care setting, various isothermal amplification techniques has been developed including strand displacement amplification (SDA), loop-mediated amplification (LAMP), nucleic acid sequence-based amplification (NASBA), helicase-dependent amplification (HDA), recombinase polymerase amplification (RPA), and others (see, e.g., Yan et al., 2014, Mol. BioSyst., 10:970-1003).

Alternatively, probe amplification methods include ligase chain reaction (LCR), Invader assay, Padlock probes, rolling circle amplification (RCA), with detection via the self-assembly of DNA probes to give supramolecular structures. Signal amplification methods, as with probe amplification methods, do not require target nucleic acid amplification and include nicking endonuclease signal amplification (NESA) and nicking endonuclease assisted nanoparticle activation (NENNA), Junction or Y-probes, split DNAZyme and deoxyribozyme amplification strategies, template-directed chemical reactions that lead to amplified signals, non-covalent DNA catalytic reactions, hybridization chain reactions (HCR), Tyramide Signal Amplification, Branched DNA (bDNA) (see, e.g., Andras et al., 2001, Mol. Biotech., 19(1):29-44; and Yan et al., 2014, Mol. BioSyst., 10:970-1003). With the progress of nanotechnologies, signal detection systems quickly adopted the use of bioconjugated nanoparticles (NPs) and a variety of detection systems were developed (see, e.g., Ju et al., 2011, Signal Amplification for Nanobiosensing, Ch. 2 in NanoBiosensing, pp 39-84).

Among the probe amplification methods, rolling circle amplification (RCA), often referred to as rolling circle replication (RCR), generates multiple copies (e.g., tandem repeats) within the same molecule, making it difficult to detect small single DNA circles from the large (e.g., hundreds of nanometers to micrometer size) coiled single molecule structures easily detectable upon hybridization of hundreds of fluorescently-labeled detector probes. Such large structures can be deposited on a solid support and counted, providing a digital read-out for detection and accurate quantification of a target nucleic acid. Conceptually, the methods described herein are similar to droplet digital PCR (ddPCR), where the single linear DNA molecules encapsulated into small reactors are specifically amplified by PCR using target-specific amplification primers. The benefit of RCA compared to ddPCR is that there is no need to compartmentalize the single DNA molecules prior to amplification, as the RCR mechanism generates long single-stranded DNA molecules spontaneously collapsing into large distinct individual structures in the same reaction volume.

In the present disclosure, the novel methods of detecting target nucleic acid include two consecutive probe ligation reactions to generate the circular probe and amplify the probe via rolling circle replication mechanism for digital detection. The methods described herein are more cost effective compared to other methods known in the art such as Padlock probes and Selector probes, which also exploit the rolling circle probe amplification, as the methods described herein can utilize much shorter oligonucleotides in the composition of the probe, which are much more easy to manufacture and cost significantly less than longer probes. Specifically, the methods described herein are very useful for detecting fetal aneuploidies in cell-free DNA samples obtained from the blood of pregnant women.

Another novel method described herein also is based on rolling circle amplification, but instead of generating and amplifying the circular probe, the fragments of nucleic acid in the sample are circularized and the target nucleic acid is selectively amplified by rolling circle replication for detection and enumeration, if desired. This method is especially well suited for detecting fetal aneuploidies in samples containing cell-free fetal DNA, as the size of the majority of cell-free DNA species in these samples is within about 165 to about 190 nucleotides, which is amenable for direct template-free circularization/ligation without the need to fragment the sample DNA.

The methods described herein are single-molecule digital detection methods, as the signal from a single probe, or a target nucleic acid molecule, becomes detectable using rolling circle amplification of a probe or a target.

In some embodiments, methods and compositions of detecting target nucleic acid in a sample that includes fragments of nucleic acid is provided, where the specificity of detection is achieved by employing target-specific probes that hybridize to the target nucleic acid and are further converted into a circular form (e.g., a target-specific circle). Each target-specific circle, which is a single molecule, is amplified via rolling circle replication to generate a long linear nucleic acid having multiple tandem repeats. Thus, this long linear nucleic acid having multiple tandem repeats becomes detectable and quantitatable upon hybridization with a probe-specific fluorescently-labeled detector-probes (see exemplary method in FIG. 9A). In some embodiments, the methods described herein are tailored for testing a sample containing cell-free circulating DNA for fetal chromosomal aneuploidies. Such methods, which includes detection and quantification of chromosome-specific fragments also are disclosed (see, e.g., FIG. 9B).

Methods and compositions for detecting target nucleic acids in a sample that includes fragments of nucleic acids are provided, where the fragments of the nucleic acids first are circularized, and then the target-specific circles are selected for amplification by rolling circle replication from the bulk circles using target-specific primers. The detection of target nucleic acid includes hybridization of fluorescently-labeled target-specific detector-probes with the products of rolling circle replication, and also can include enumerating the dye-specific products (see, e.g., FIG. 9C). These methods also can be tailored for testing a sample having cell-free circulating DNA for fetal chromosomal aneuploidies. Such methods, which includes detection and quantification of chromosome-specific fragments, also are disclosed (see, e.g., FIG. 9D).

### 2. Target Nucleic Acid Detection and Quantification Using A Probe

The methods of target nucleic acid detection described herein include a first ligation of the arms of the probe using target nucleic acid as a template, and a second ligation to circularize the ligated probe (see, e.g., FIG. 10A). Prior to contact the nucleic acid in the sample with the probe, the nucleic acid in the sample is denatured, e.g. by heating and snap cooling. A probe can include a left arm oligonucleotide 200 and a right arm oligonucleotide 201 (referring to FIG. 10). The left arm of the probe includes a 15-35 nucleotide long moiety 202, which is complementary to upstream sequences in a target nucleic acid fragment 208, and a custom non-complementary moiety 204 having a portion complementary to a detector-probe 206 and a portion 207 partially complementary to the 5′ portion of an oligonucleotide serving as a primer for rolling circle replication 212. The right arm of the probe includes a 15-35 nucleotide long moiety 203, which is complementary to downstream sequences in a target nucleic acid fragment 208, and a custom non-complementary moiety 205 that includes a short sequence of 2-5 nucleotides complementary to the 3′ end portion of the oligonucleotide 212, which serves as a primer for rolling circle replication.

Under conditions in which the left and right arms of the probe anneal to the target nucleic acid 208, the target-complementary moieties 202 and 203 form a double-stranded complex such that the 3′ end 202 is in juxtaposition to the 5′ phosphorylated end 203, and the two ends are ligated with the aid of DNA ligase (e.g., T4 DNA ligase or Taq Ligase). This generates a product of ligation that is a continuous linear strand of nucleic acids. After ligation, the double-stranded complexes of ligated probe and target nucleic acid are denatured by heating and circularized with the aid of single-stranded DNA ligase, e.g. CircLigase II, to produce covalently closed single-stranded circles 209, where the integrity of the sequence complementary to the primer of rolling circle replication 212 is restored. Since the nucleic acid fragments in the sample were treated with phosphatase to remove 5′ end phosphate groups, they can't be circularized. The unreacted left and right arms of the probe also can be circularized, as they have phosphate groups at their 5′ ends 210 and 211.

After digestion of the linear polynucleotides in the ligation mix using an exonuclease, e.g., Exonuclease I, a probe-specific primer 212 can be annealed to the circles. Annealing the primer 212 to the circularized probe 209 produces a complex capable of initiating rolling circle replication by DNA polymerase having strand-displacement activity, e.g. Phage φ29 DNA polymerase, as the 3′ end of the primer is perfectly annealed to the circle. Conversely, annealing the primer 212 to the circularized left arm of the probe 210 produces a complex incapable of initiating DNA synthesis, as the 3′ end of the primer 212 does not match the circular template.

To prevent digestion of the 3′ end of the primer 212 by the 3′-5′ exonuclease activity of Phage φ29 DNA polymerase, which may result in potential priming of circles 210 and other non-specific priming, a phosphorothioate backbone resistant to 3′-5′ exonuclease action can be introduced into the last 1-3 nucleotide linkages of the primer oligonucleotide 212. The circularized right arm 211 then is unable to form a stable complex with the primer 212, and, therefore, is incapable of priming and initiating rolling circle replication. Thus, only one product of circularization, the full-length circularized probe 209, serves as the template for rolling circle replication and produces long single-stranded molecules having the tandem repeated copies of the probe.

In some embodiments, a probe can be designed to discriminate and detect small genetic variations in the genome, e.g., a single nucleotide polymorphism (SNP) or a mutation in a germ line or in cancer cells. Changing, by design, the type of nucleotide at the 3′ end in the left arm moiety 202, or at the first 5′ nucleotide position in the right arm moiety 203, allows for two alleles to be discriminated because a DNA ligase, e.g. T4 DNA ligase, under standard conditions can ligate predominantly only DNA ends that are perfectly matched to the template strand on both flanks of the ligation junction. Thus, depending of the presence of a match or a mismatch next to the ligation point, moieties 202 and 203 can be joined together, or not, by a DNA ligase.

In some embodiments, the method of target nucleic acid detection includes a first ligation of the arms and an additional oligonucleotide, a “bridge”, of the probe using target nucleic acid as a template and a second ligation to circularize the ligated probe (FIG. 10B). Compared to the method shown in FIG. 10A, the probe in FIG. 10B also includes a bridge oligonucleotide 213 in addition to the left arm oligonucleotide 200 and the right arm oligonucleotide 201. As used herein, a bridge oligonucleotide 213 is complementary to a central region of the target nucleic acid between the upstream and downstream sequences that are complementary to the moieties of the probe 202 and 203. The addition of a bridge oligonucleotide to a probe not only further increases the specificity of the assay, as two ligation events are required to generate the ligated linear probe polynucleotide, but also provides a way to better interrogate small genetic variations in a target nucleic acid, e.g. SNPs—single nucleotide polymorphisms. The bridge oligonucleotide 213 can be 5-10 nucleotides long and nucleotide variation can be placed in various positions along the length of the bridge oligonucleotide, including at the 5′or 3′ ends and/or next to the ligation junction with the left or right arm of the probe. The shorter the bridge oligonucleotide, the better the discrimination is between matches and mismatches, as even one mismatch within a 5-6 nucleotide long bridge has a significant effect on the stability of the double-stranded complex that is formed between the bridge and its complementary target sequence.

In another aspect, methods are provided (FIG. 11A and FIG. 11B) where the second ligation (the circularization of the probe) is a template-dependent ligation (i.e., where the ligation junction is within the region of double-strandedness compared to the embodiment shown in FIG. 10). In the embodiment shown in FIG. 11, there is no need to split the sequence 307 that is complementary to the primer for rolling circle replication 311 as is required in the method of FIG. 10, where the restoration of the integrity of the sequence 207 serves as a selective mechanism to amplify, using RCR, only the circles that underwent two consecutive ligations, and to discriminate against circularized single arms of the probe. The template-dependent intramolecular ligation can be used as a second ligation reaction to form target-specific circles, compared to the method shown in FIG. 10 in which template-independent ligation of the single-stranded 5′ and 3′ ends is performed using a single-stranded DNA ligase.

As depicted in FIG. 11A and 11B, only the ligated probes 314 and 315 having a left arm 300 and a right arm 301 (referring to FIG. 11A, or the left arm 300, the right arm 301, and the “bridge” 309 referring to FIG. 11B) can be circularized using a splint oligonucleotide 310. A splint oligonucleotide 310 as used herein is complementary to the custom 5′ end sequences of the left arm and the 3′ end sequences of the right arm. Under conditions promoting the annealing of these ends to the splint oligonucleotide 310, a double-stranded DNA complex is formed, where the 5′ phosphorylated end of the custom moiety 303 of the left arm 300 is in juxtaposition to the 3′ end of the custom moiety 306 of the right arm 301, and the two ends can be ligated using a DNA ligase (e.g. T4 DNA ligase, or Taq Ligase). This results a product of ligation 312 that includes covalently-closed circular single-stranded DNA. The method shown in FIG. 11B is different from the method shown in FIG. 11A only in one aspect—the “bridge” oligonucleotide 309 in FIG. 11B is used to further increase the specificity of the formation of ligated linear probe 315, where two ligation junctions are required to be formed. The splint-assisted ligation-circularization results in formation of covalently closed single-stranded DNA circles 313. Additionally, the use of the “bridge” further increases the specificity and flexibility to interrogate small genetic variations in a target nucleic acid, as discussed herein.

In some embodiments, each of the plurality of target-specific primers and each of the plurality of detector-probes is from 10 to 200 nucleotides in length.

### 3. Target Nucleic Acid Detection and Quantification Using the Target-Specific Priming of Circularized Sample Nucleic Acid

FIG. 12 shows another embodiment of the methods described herein for detecting target nucleic acids in a sample that includes circularization of fragmented nucleic acid, e.g. DNA. For example, FIG. 12 depicts the concurrent detection of two nucleic acid targets. Such methods exploit the property of single-stranded DNA ligases to efficiently circularize the fragments of single-stranded DNA (ssDNA). Commercially available ssDNA ligases include, without limitation, CircLigase™, CircLigase II™ (Epicentre), and Thermophage Ligase (Prokaria). For example, CircLigase™ II ssDNA Ligase is a thermostable enzyme and can catalyze intramolecular ligation (i.e., circularization) of ssDNA templates having a 5′-phosphate and a 3′-hydroxyl group. It can be used to make circular ssDNA molecules from linear ssDNA fragments. Such ssDNA circles serve as templates for rolling-circle replication, or rolling-circle transcription. In contrast to T4 DNA Ligase, which ligates DNA ends that are annealed adjacent to each other on a complementary DNA template, the CircLigase II ssDNA Ligase ligates the ends of ssDNA in the absence of a complementary sequence. Linear ssDNA of >15 bases, including cDNAs, are circularized by CircLigase II enzyme. Under standard reaction conditions, virtually no linear concatemers or circular concatemers are produced. Circularization efficiency decreases with the increasing the length of ssDNA, e.g., circularization is not efficient for templates >500 bases in length. There, such a method as disclosed herein typically uses fragmented DNA, or cDNA, in the range of 50-500 nucleotides in length.

In some embodiments, the ssDNA of this size range can be generated by fragmentation of sample dsDNA physically (e.g. sonication), or enzymatically (e.g. NEBNext® dsDNA Fragmentase, New England Biolabs Inc.). In some embodiments, cDNA generated from fragmented RNA can be used as the template for circularization. In some embodiments, the circulating cell-free DNA (ccfDNA) found in a human blood sample is used. This DNA can originate from the normal cells of the host, from fetal DNA, or from tumor DNA. The ccfDNA is represented mainly by dsDNA fragments of ˜170 base pairs. The size of such fragments corresponds approximately to the size (length) of dsDNA wrapped around a nucleosome. After denaturation, the ssDNA fragments of ˜170 nucleotides are well-suited for efficient circularization by CircLigase II.

In the methods disclosed herein, the fragments of dsDNA present in the sample are first converted into single-stranded form by either heat denaturation/quick chill on ice, or an alkali denaturation/neutralization method, and then circularized, e.g., by CircLigase II, to generate ssDNA circles. Depending on the DNA shearing method, dsDNA fragments may need to be repaired to restore the 5′ phosphate and the 3′ hydroxyl groups required for ligation. This can be accomplished by the treatment of dsDNA with polynucleotide kinase. dsDNA fragments generated enzymatically, e.g., by NEBNext® dsDNA Fragmentase, do not need to undergo end repair.

In some embodiments, after ssDNA circularization, the uncircularized linear fragments can be digested with exonuclease, e.g. Exonuclease I from E. coli, to enrich for ssDNA circles. This prevents any potential non-specific priming of rolling circle replication by uncircularized linear fragments and ensures that target-specific primers initiate DNA synthesis only on circles.

In some embodiments, at least one target-specific rolling circle replication primer is provided to initiate rolling circle replication. In some embodiments, a plurality of primers, which may be referred to as a set or primers, that are complementary to a target nucleic acid. For example, when the genome of a pathogen or a virus, or a chromosome or chromosome locus is used, a set of primers can include at least 10 different primers (e.g., at least different 100 primers, or at least 1000 different primers) that each specifically recognize a distinct target sequence.

In some embodiments, rolling circle replication is initiated from the primer-circle complexes by a DNA polymerase possessing strong strand-displacement activity, which is a prerequisite of processive rolling circle replication mechanism. “Strand displacement” describes the ability of an enzyme to displace downstream DNA encountered by the enzyme during synthesis. Exemplary DNA polymerases include Phage φ29 DNA polymerase, Bst DNA polymerase Large fragment, and Bsu DNA polymerase Large fragment. Phage φ29 DNA polymerase has the strongest strand-displacement activity and is active at moderate temperatures (e.g., around 20-37° C.). Bsu DNA polymerase Large fragment has a moderate strand-displacement activity and is active at moderate temperatures (e.g., 20-37° C.). Bst DNA Polymerase, Large Fragment, on the other hand, is a good strand-displacing enzyme that is active at elevated temperatures (e.g., around 65° C.). Rolling circle replication uses a rolling circle, a replicative structure in which one strand of a circular duplex is used as a template for multiple rounds of replication, generating many copies of that template. A rolling circle is formed when DNA synthesis is initiated from the 3′ end of the primer and dsDNA is generated until the polymerase enzyme reaches the 5′end of the annealed primer. The DNA polymerase then begins to displace the upstream 5′end. The newly synthesized strand displaces the original nicked strand, which does not serve as a template for new synthesis. Thus, the rolling circle mechanism copies only one strand of the DNA. Elongation proceeds by replication going around the template multiple times, in a pattern resembling a rolling circle. This results in the production of a large number of copies of a single strand of a DNA, concatenated or connected end-to-end. Rolling circle replication driven by Phage φ29 DNA polymerase generates linear concatemeric products of >50 kilobases long in just 20-30 minutes. Such long linear molecules fold into micrometer-sized random coils, which can be detected upon deposition on a surface and staining by hybridization of fluorescently-labeled probes.

In some embodiments, the methods disclosed herein include performing concurrent rolling circle replication of multiple targets present within a mix of ssDNA circles. Such a strategy is termed “multiplexed target detection”, or “multiplexing”. Such a multiplexed target detection can be performed using multiple primer sets, where each of the multiple primer sets includes a plurality of primers specifically recognizing a distinct target sequence. Multiple primer sets includes at least two primer sets. The number of primer sets is limited only by the number of optically resolvable sets of corresponding detector-probes. FIG. 12 depicts concurrent interrogation of two distinct targets in a nucleic acid sample, which includes the initiation of rolling circle replication with two distinct target-specific primer sets, where, for simplicity, each primer set is represented by one single primer.

In some embodiments, the priming of rolling circle replication includes hybridization of primers to the ssDNA circles. This can be accomplished by heating the mix of circles and primers above 90° C. followed by a slow cooling to a reaction temperature of about 30-37° C., and subsequent addition of Phi29 DNA polymerase, buffer, and co-factors to initiate DNA synthesis.

In some embodiments, the primers include oligonucleotides having a hairpin (or a stem-loop) structure similar to molecular beacon probes, but without a fluorophore moiety attached. For example, the sequence at the 3′ end of the primers can be complementary to the target and 5-8 nucleotides complementary to the 3′ end can be added to the 5′ end of the primers. Hairpin primers increase the specificity of rolling circle replication, because, at the temperatures of rolling circle replication (e.g., 30-37° C.), such primers are in the stem-loop form in which the 3′ ends are hidden within the double-stranded stem and can't participate in erroneous DNA synthesis to form primer-dimers or non-specific RCR products, the two major spurious products of rolling circle replication that are produced at moderate reaction temperatures.

To enhance the specificity of the rolling circle replication, the primers can include oligonucleotides with blocked-cleavable 3′ ends, similar to rhPCR (RNase H-dependent PCR) primers developed by IDT (see, e.g., Dobosy et al., 2011, BMC Biotechnol., 11:80). Such primers include, in the 3′ to 5′ direction, the 1^{st} DNA base that is a mismatch to the target, an RNA base that matches the target located in the 6^{th} position, two matching DNA bases at the 2^{nd} and 5^{th} positions, and blocking groups (C3 spacers) in the 3^{rd} and 4^{th} positions. Such primers are non-functional prior to being unblocked by RNase HII. After annealing of such primers to ssDNA circles, unblocking is achieved by RNase HII-mediated cleavage at a single RNA residue placed near the 3′ end of the primer. Cleavage removes the blocking group, leaving a 3′-OH end capable of priming DNA synthesis. Cleavage is sensitive to match/mismatch at and around the RNA base position, resulting in very high specificity for base sequence in this region.

Rolling circle replication can be stopped by the addition of ethylenediaminetetraacetic acid (EDTA). The length of the rolling circle replication products is reaction time-dependent in approximately a linear manner. Hence, the folding of replication products into random coils results in the formation of structures having a certain diameter. For example, the concatemeric coil can have a cross-sectional diameter of at least 50 nanometers (e.g., at least 100 nanometers, at least 500 nanometers, at least 800 nanometers, at least 1 micrometer, at least 2 micrometers or greater).

### 4. Deposition of RCR Products to the Surface, Detection, and Enumeration

The products of rolling circle replication can be deposited on a surface (e.g., a solid, semi-solid, or gel surface). A solid support can include, but is not limited to, materials such as glass, polyacryloylmorpholide, silica, controlled pore glass, polystyrene, polystyrene/latex, carboxyl modified Teflon, polymerized Langmuir Blodgett film, functionalized glass, Si, Ge, GaAs, GaP, SiO2, SiN4, modified silicon, or (poly) tetrafluoroethylene, (poly) vinylidendifluoride, polystyrene, polycarbonate, or combinations thereof. Solid supports include, but are not limited to, slides, plates, beads, particles, spheres, strands, sheets, containers (e.g., test tubes, microfuge tubes, trays and the like), capillaries, films, polymeric chips and the like. The surface of the substrate can be planar. Regions on a substrate can be physically separated from one another, for example, using trenches, grooves, wells or the like. Semi-solid supports can be selected from polyacrylamide, cellulose, polyamide (nylon) and crossed linked agarose, dextran and polyethylene glycol.

In some embodiments, a support can include a variety of different binding moieties to allow concatemers to be coupled to the support. A suitable binding moiety includes, but is not limited to, a capture moiety such as a hydrophobic compound, an oligonucleotide, an antibody or fragment of an antibody, a protein, a chemical cross-linker, one or more elements of a capture pair, e.g., biotin-streptavidin, NETS-ester and the like, a thioether linkage, static charge interactions, van der Waals forces or the like. The support can be functionalized with any of a variety of functional groups known in the art. Commonly used chemical functional groups include, but are not limited to, carboxyl, amino, hydroxyl, hydrazide, amide, chloromethyl, epoxy, aldehyde and the like.

In some embodiment, the products of rolling circle replication are deposited on the surface of an aminosilane-coated glass slide or in wells in a 96-well plate.

In some embodiments, after deposition of the products of rolling replication on the surface of a substrate, they can be detected and visualized by contacting them with target-specific detector-probes. Detector-probes, or detector-probe sets, as exemplified in FIG. 12 (method of FIG. 9C), are differentially labeled with fluorescent dyes. Upon hybridization, two species of products become distinguishably labelled, and the number of products corresponding to the two targets can be counted. The same detection strategy also can be used in the methods shown in FIG. 9A, 9B, and 9C. The major difference between the methods shown in FIG. 9A and 9B and the methods of FIG. 9C and 9D is that the detector-probes for the first two methods are complementary to the custom moiety of the ligation probes, e.g., 206 in FIG. 10A and 10B and 304 in FIG. 11, while in the last two methods, the detector-probes are complementary to the target DNA molecules in their linear form present in the sample and in the corresponding circular form.

For quantifying relative amounts of multiple species of nucleic acids, different signals can be used for each species, for example, the products of one set of probes can emit a different wavelength or spectrum of fluorescence compared with the products of another set of probes. In some embodiments, the methods described herein can be tailored for a sample containing cell-free circulating DNA for testing for fetal chromosomal aneuploidies (see, e.g., the methods in FIG. 9B and FIG. 9D). Such methods can include a) depositing a mixture of first and second RCR products, corresponding to the chromosome being tested for aneuploidy and a control chromosome, to the surface of a substrate; b) hybridizing with a first and second plurality of detector-probes, where the first and the second plurality of detector-probes are distinguishably labeled; and c) counting the number of RCR products bound to the first plurality of detector-probes, and independently, counting the number of RCR products bound to the second plurality of detector-probes. Since each RCR product includes hundreds or thousands of copies of the original single-stranded DNA circle, the labeled detector-probes hybridize to these multiple repeated sequences to produce strong fluorescent signal easily detectable by optical imaging systems, thus, allowing the accounting of each RCR product. Such a digital counting of signals from individual RCR products allows accurate counting of DNA fragments corresponding to particular chromosome (e.g. chromosome 21 vs. control chromosome 1) present in the sample of cell free DNA. The methods described herein are more accurate than PCR, because amplification of the target molecules can result in significant bias, as some sequences are amplified at much higher efficiencies than others.

In some embodiments, detector-probes can be fluorescently labeled. Suitable distinguishable fluorescent label pairs useful in the methods described herein include Cy-3 and Cy-5 (Amersham Inc., Piscataway, N.J.), RadiantDY-547 and RadiantDY-647 (BioVentures, Inc., Murfreesboro, Tenn.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.). It would be understood that more than two fluorescent labels are required for multiplexed target detection. Suitable distinguishable detectable labels may be found in the web resource “Database on Fluorescent Dye” (fluorophores.org on the World Wide Web) and in Kricka et al. (2002., Ann. Clin. Biochem., 39:114-29).

As there are a limited number of spectrally distinct fluorophores to be used simultaneously, multiplexing currently is limited to 5-6 dyes. However, a combinatorial labeling method can be used to generate the required number of different emission spectra. In some embodiments, for multiplexed detection higher than 5-plex, the target-specific detector-probes can include the probes labeled with fluorescent dyes according to “Multicolor Combinatorial Probe Coding” (MCPC) (see, e.g., Huang et al., 2011, PLoS ONE, 6(1):e16033), which describes a labeling paradigm that uses a limited number (n) of differently colored fluorophores in various combinations to label each probe, enabling all of 2ⁿ-1 targets to be detected in one reaction. It would be appreciated that RCA products can be labeled before or after they are distributed on the substrate.

In some embodiments, the methods described herein typically include splitting the sample, which includes circularized DNA fragments, into several reactions prior to initiation of RCR, as depicted in FIG. 13A for 2-plex target detection assay as an example, where the two RCR reactions are performed in separate compartments using polymerase and dNTPs, and where the nascent RCR products are labeled during the chase reaction with dye-labeled dNTPs to generate distinguishably labeled target-specific RCR products. Alternatively, the RCR reaction can include labeled dNTPs at initiation, as depicted in FIG. 13B. In this case, the ratio of dye-labeled dNTPs to unlabeled dNTPs is the main factor determining the balance between the density of incorporation of dye-labeled dNTPs and the overall length of the RCR product. A very high ratio may limit the progress of rolling circle replication and result in abortive RCR due to distortion of the DNA helix by incorporation of bulky dye moieties, ultimately causing an inability of DNA polymerase to perform further DNA synthesis. Feasibility of the dye-labeled dNTP incorporation during rolling circle replication has been demonstrated (see, e.g., Smolina et al., 2005, Analyt. Biochem., 347:152-5). Fluorescently labeled dNTPs are commercially available, e.g., Cy3.5-dCTP (GE Healhcare Life Sciences); 5-FAM-dUTP, Andy Fluor 488-X-dUTP, Andy Fluor 555-X-dUTP, Andy Fluor 568-X-dUTP, Andy Fluor 594-X-dUTP, Andy Fluor 647-X-dUTP, Cy3-X-dUTP, Cy5-X-dUTP (Applied BioProbes); Diethylaminocoumarin-5-dUTP, Diethylaminocoumarin-5-dCTP, Cy5-dGTP, Cy5-dUTP, Cy5-dATP, Cy5-dCTP, Texas Red-5-dUTP, Texas Red-5-dCTP, Texas Red-5-dATP, Cy3-dUTP, Cy3-dCTP, Cy3-dGTP, Cy3-dATP, Tetramethylrhodamine-6-dUTP, Tetramethylrhodamine-6-dCTP, Lissamine-5-dUTP, Fluorescein-12-dUTP, Fluorescein-12-dCTP, Fluorescein-12-dGTP, Fluorescein-12-dATP (Perkin Elmer). Such RCR product labeling methods, which are alternatives to the hybridization of fluorophore-labeled detector-probes (see, e.g., FIG. 12), can result in incorporation of hundreds or thousands of fluorophores in each RCR product for easy signal detection.

In some embodiments, the distinguishably labeled split RCR reactions are pooled back together prior to deposition on the surface for quantification (see FIG. 13A and 13B), or deposited on separate areas on the surface of support (FIG. 14A and 14B). In the latter case, the split reactions can be labeled during RCR by incorporation of the same fluorophore.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Part A—Methods of Detecting Target Nucleic Acids Using a Positively Charged Tag

### Example 1

### Cleavage of an Oligospermine-Oligonucleotide Conjugate Probe

A target polynucleotide detection probes comprising Zip Nucleic Acid (ZNA) oligonucleotide was synthesized, where the 5′ or 3′ detectable tag is an oligospermine polycationic 5′ or 3′ tail having 3 or 4 spermine units with the net charge of (−++++)×3=9⁺, or (−++++)×4=12⁺, respectively.

Probe 1 for RNaseP qPCR assay:
(SEQ ID NO: 1)
5′-(Spermine)₃-(Spacer-31)-TTC TGA CCT GAA GGC TCT
GCG CG-(Spacer-C3)-3′
Probe 2 for RNaseP qPCR assay:
(SEQ ID NO: 2)
5′-TTC TGA CCT GAA GGC TCT GCG CG-(Spacer-31)-
(Spermine)₄

The oligonucleotide portion of the probe is a sequence complementary to the human RNaseP gene. The 3′ end of Probe 1 is blocked by a C3 spacer to prevent priming of DNA synthesis by DNA polymerase from the 3′ end of the annealed probe. PCR forward and reverse primers were designed to generate a RNaseP amplicon of 60 bp.

Forward primer for RNaseP qPCR assay:
(SEQ ID NO: 3)
5′-AGA TTT GGA CCT GCG AGC G-3′
Reverse primer for RNaseP qPCR assay:
(SEQ ID NO: 4)
5′-GAG CGG CTG TCT CCA CAA GT-3′

Probe 1 is expected to be cleaved during PCR by the 5′ exonuclease (flap endonuclease) activity of the Taq polymerase to release the 5′ polycationic oligospermine tag. The oligonucleotide portion of Probe 2 is expected to be digested during PCR by the 5′ exonuclease activity of the Taq polymerase to release the 3′ polycationic oligospermine tag.

The PCR amplification of 60 bp amplicon of human RNaseP gene was performed in the presence of either Probe 1 or Probe 2 under the reaction conditions specified below. PCR was performed in reaction mixes containing 1× Taq Buffer (New England Biolabs Inc.), 200 μM of each dNTP, 0.8 μM Forward primer, 0.8 μM Reverse primer, 0.4 μM probe, 5 U/μl Hot Start Taq (New England Biolabs Inc.), and 100 ng human DNA. Six PCR reactions were set up as follows:

Reaction 1: PCR using Probe 1;

Reaction 2: “No-Template-Control” (NTC)—same as Reaction 1, but no template DNA was included;

Reaction 3: PCR using Probe 2;

Reaction 4: “No-Template-Control” (NTC)—same as Reaction 3, but no template DNA was included;

Reaction 5: “Negative control”—reaction mix with Probe 1, but Forward primer, Reverse primer, Taq polymerase, and template DNA were omitted,

Reaction 6: “Negative control”—reaction mix with Probe 2, but Forward primer, Reverse primer, Taq polymerase, and template DNA were omitted.

Thermocycling was performed under the following conditions: 95° C. for 30 sec; 30 cycles of 95° C. for 15 sec, 55° C. for 30 sec, 68° C. for 30 sec; and 68° C. for 5 min. After the completion of PCR, the reactions were loaded on a 15% TBE-urea gel, run at 180 V, and stained with GelStar intercalating dye (Lonza, Inc.). FIG. 8 demonstrates that Probe 1 is specifically cleaved during the PCR reaction (Lane 1), as compared to the NTC (Lane 2). Lane 5 shows the electrophoretic mobility of Probe 1. Similarly, Probe 2 is cleaved only in the sample where successful amplification of the product occurred (Lane 3), and no cleavage is observed in the control NTC sample (Lane 4). Lane 6 shows the electrophoretic mobility of Probe 2. These results demonstrate that the probes with the positively charged tags at the 5′-, or 3′-end can be efficiently cleaved by Taq polymerase.

### Part B—Methods of Detecting Target Nucleic Acids Using Rolling Circle Replication

### Example 1

### Detection of Trisomy 21 (Down Syndrome)

An exemplary method, as depicted in FIG. 11A, was design to interrogate human chromosome-specific regions. 192 human Chromosome 1-specific and 192 human Chromosome 21-specific probes were synthesized. Each probe included two 5′ phosphorylated oligonucleotides: a left arm and a right arm. Each left and right arm oligonucleotide had a 20-30 nt region of homology to targeted human sequences, and a custom sequence flap having the sequence: 5′-TCG ACC GAC CAC CCT AGC GAC CCG TA-3′ (SEQ ID NO:5) for the left arm of Chromosome 1 probes, 5′-TCG ACC GAC CCT TCT GAG CTC CTG CG-3′ (SEQ ID NO:6) for the left arm of Chromosome 21, and 5′-GCC CGA CTT AGC GTA CCA-3′ (SEQ ID NO:7) for the right arms of both Chromosome 1- and Chromosome 21-specific probes.

Human DNA of NA19238 female from Coriell Cell Repository (Camden, N.J.) was fragmented with Fragmentase (New England Biolabs) and size selected to a mean fragment size of 180 nt. 20 ng of fragmented DNA was combined with the mix of two pools of chromosome specific probes, each consisting of 192 probes (each of 384 probes is at a final concentration of 5 nM), annealed and ligated at 62° C. by HiFi Taq Ligase according to manufacturer conditions. The single-stranded products of ligation, having a mean size of ˜90 nt, were separated from the probes by gel electrophoreses, cut out and extracted from the 2% gel using Nucleospin Kit (Machereu-Nagel). Ligation products in 60 μl of 10 mM Tris pH 8/0.1 mM EDTA were mixed with 10 μl of 10 μM Splint oligonucleotide (5′-GGT CGG TCG ATG GTA CGC TAA GTC-3′ (SEQ ID NO:8)), heated to 95° C. for 3 min, then snap-cooled on ice. 50 ul of ligation mix consisting of 1× TA buffer (33 mM Tris-acetate (pH 7.5), 66 mM potassium acetate, 10 mM magnesium acetate, and 0.5 mM DTT), 1 mM ATP, and 2.1 Units/μl of T4 DNA ligase was added and ligation/circularization reaction was carried out for 1 hour at 37° C. Exonuclease I and Exonuclease III (both from Enzymatics Inc) were added to the ligation mix to the final concentration of 0.62 Units/μl and 1.03 Units/μl, respectively, and incubated in a thermal cycler at 37° C. for 0.5 hour. To stop the reaction, 6 μl of 0.5 M EDTA was added to the samples. Single-stranded DNA circles were purified using Nucleospin Clean-Up Kit (Macherey-Nagel), and eluted in 20 μl of 10 mM Tris pH 8. RCR primer (same as Splint oligonucleotide) was added to a final concentration of 0.5 μM and the 30 μl reaction mix was heated to 93° C. for 3 min and slowly cooled down to 30° C. over 30 mins. Rolling Circle Replication was set up by adding the following reaction components to the mix containing Circles with annealed RCR primer: 1× Phi29 buffer, 0.2 mM dNTP (each), 0.2 μg/μl BSA, 200 mU/μl Phi29 DNA polymerase (New England Biolabs). The reaction was performed at 30° C. for 1 hour and stopped by the addition of EDTA. The final RCR products were deposited for 30 min on the surface of amino-silane glass slides in a custom flow cell, and hybridized with the mix of Chromosome 1-specific (5′-Cy3-CCA CCC TAG CGA CCC GTA-3′ (SEQ ID NO:9)) and Chromosome 21-specific (5′-Cy5-CCC TTC TGA GCT CCT GCG-3′ (SEQ ID NO:10)) in hybridization buffer (1 M NaCl/20 mM EDTA/20 mM Tris/0.1% Tween-20 for 2 min at 70° C. and 30 min at 50° C.) at final decorator probe concentration of 5 nM. Flow cell was washed with hybridization buffer and Cy3 and Cy5 fluorescence images of the same field of view were taken to visualize RCR products specific for Chromosome 1 and 21. FIG. 15.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of amplification-free target nucleic acid sequence detection, the method comprising:
(a) providing a sample comprising at least one target nucleic acid sequence;
(b) contacting the sample with a probe comprising a nucleic acid moiety and a positively-charged tag, wherein the nucleic acid moiety is complementary to at least a portion of the target nucleic acid sequence, wherein the contacting is performed under conditions in which a probe-target complex is formed between the nucleic acid moiety and the complementary target nucleic acid sequence;
(c) cleaving the probe within the probe-target complex to release the detectable positively-charged tag; and
(d) detecting the movement of the released positively-charged tag through a nanopore based on a change in an electrical signal;
wherein a change in the electrical signal indicates the presence of the target nucleic acid sequence in the sample.

2. The method of claims 1, wherein the probe further comprises a scissile linkage selected from the group consisting of RNA sequences, DNA sequences, and abasic nucleotide sequences.

3. The method of claim 2, wherein the scissile linkage comprises at least one RNA residue.

4. The method of claim 2, wherein the scissile linkage comprises an oxidized purine or an oxidized pyrimidine.

5. The method of claim 2, wherein the scissile linkage comprises an apurinic site or an apyrimidinic site.

6. The method of claim 2, wherein the scissile linkage comprises deoxyuridine, 5-hyroxyuracil, 5-hydroxymethyluracil, or 5-formyluracil.

7. The method of claim 2, wherein the scissile linkage is cleaved by type 1 ribonuclease H or type 2 ribonuclease H.

8. The method of claim 2, wherein the scissile linkage is cleaved by a combination of DNA N-glycosylase and DNA AP-lyase activity.

9. The method of claim 2, wherein the scissile linkage is cleaved by DNA AP-lyase activity or an endodeoxyribonuclease.

10. The method of claim 2, wherein the scissile linkage is cleaved by a combination of DNA N-glycosylase and endodeoxyribonuclease, or a combination of DNA N-glycosylase and DNA AP-lyase activity.

11. The method of claim 1, the method further comprising:
in step (b), further contacting the sample with a primer, wherein the primer is complementary to at least a portion of the target nucleic acid sequence that is upstream of the portion of the target nucleic acid sequence to which the probe is complementary, wherein the contacting is performed under conditions in which a primer-target complex is formed between the primer and the complementary target nucleic acid sequence; and
in step (c), contacting the sample comprising the primer-target complex with DNA polymerase under conditions in which primer extension occurs.

12. The method of claim 11, wherein the probe is cleaved during primer extension by the DNA polymerase.

13. The method of claim 11, wherein the cleavage is mediated by the 5′ nuclease activity of the DNA polymerase.

14. The method of claim 11, wherein the extension by the DNA polymerase is limited by the number of dNTP types present in the reaction.

15. The method of claim 14, wherein the number of dNTPs present in the reaction is one, or two, or three of the four dNTPs.

16. The method of claim 1, the method further comprising:
in step (b), further contacting the sample with at least two primers, wherein the at least two primers are complementary to portions of the target nucleic acid sequence that flank the portion of the target nucleic acid sequence to which the probe is complementary, wherein the contacting is performed under conditions in which primer-target complexes are formed between the at least two primers and the complementary target nucleic acid sequences; and
in step (c), amplifying the target nucleic acid sequence between the at least two primers using a DNA polymerase.

17. The method of claim 16, wherein amplifying the target nucleic acid sequence comprises a polymerase chain reaction (PCR) or an isothermal reaction.

18. The method of claim 16, wherein the probe is cleaved by the DNA polymerase during amplification.

19. The method of claim 16, wherein the cleavage is mediated by the 5′ flap endonuclease activity of the DNA polymerase.

20. The method of claims 16, wherein cleavage results in detection of the detectable positively-charged tag, which is indicative of the replication of target amplicon.

21. The method of claim 1, wherein the probe comprises a net negative charge.

22. The method of claim 1, wherein the detectable positively-charged tag comprises a net positive charge before and after being released.

23. The method of claim 1, wherein the detectable positively-charged tag comprises a positively charged nucleic acid moiety, a non-nucleic acid moiety, or a combination thereof before and after being released.

24. The method of claim 1, wherein the contacting step comprises contacting the sample with a plurality of probes that each are complementary to at least two different target nucleic acid sequences and that each have a different positive charge (amount or type), and wherein the electrical signal (type/amount) is able to distinguish the at least two different target nucleic acid sequences.

25. The method of claim 1, wherein the cleaving step comprises cleaving the probe enzymatically.

26. The method of claim 1, wherein the detectable positively-charged tag passes through the nanopore.

27. The method of claim 1, wherein the detectable positively-charged tag is detectable by its charge, shape, size, or any combination thereof.

28. The method of claim 1, wherein the detecting step further comprises identifying the detectable positively-charged tag.

29. The method of claim 28, further comprising correlating the identified tag with the presence of the corresponding target nucleic acid sequence.

30. The method of claim 1, further comprising correlating the amount/level of electrical signal with the amount of the target nucleic acid sequence in the sample.

31. The method of claim 1, wherein the method uses a computer processor.

32. The method of claim 1, wherein the detectable positively-charged tag is detected using an ion-sensitive field-effect transistor.

33. A method of detecting a target nucleic acid in a sample using a target-specific probe, the method comprising:
(a) providing a sample comprising a plurality of single-stranded nucleic acid fragments;
(b) circularizing, intra-molecularly, the single-stranded nucleic acids to produce single-stranded circles;
(c) contacting the single-stranded circles with at least one probe-specific oligonucleotide primer under hybridization conditions in which the at least one probe-specific oligonucleotide primer hybridizes to the complementary sequence in the single-stranded circles and forms double-stranded primer-circle complexes;
(d) contacting the double-stranded primer-circle complexes with an enzyme under conditions in which rolling circle replication occurs;
(e) contacting the products of the rolling circle replication with a target-specific dye-labeled detector-probe under conditions in which the target-specific dye-labeled detector-probe hybridizes to the complementary sequence in the products of the rolling circle replication; and
(f) detecting the target-specific dye-labeled detector-probe, wherein the presence of the target-specific dye-labeled detector-probe indicates the presence of the target nucleic acid in the sample.

34. The method of claim 33, wherein the target specific probe is bound to a solid support.

35. The method of claim 33, wherein the circularization step is mediated by a single-stranded DNA ligase.

36. The method of claim 33, further comprising enzymatically digesting uncircularized linear nucleic acids to enrich for single-stranded circles.

37. The method of claim 33, further comprising depositing the products of the rolling circle replication on a solid support.

38. The method of claim 33, wherein the detecting step is performed using imaging.

39. The method of claim 33, wherein the detecting step comprises depositing the product of rolling circle replication on the surface of a solid support.

40. The method of claim 33, further comprising quantitating the target-specific dye-labeled detector probe and correlating the amount of target-specific dye-labeled detector probe with the amount of the target nucleic acid in the sample.

41. The method of claim 33, wherein the method is used for prenatal testing for detection of fetal aneuploidies, the method further comprising:
wherein the plurality of single-stranded nucleic acid fragments in the sample comprises fetal and maternal cell-free genomic DNA;
wherein the at least one target-specific probe comprises a plurality of chromosome-specific probes, wherein the plurality of chromosome-specific probes comprises a first set of probes comprising at least 100 different nucleic acid sequences corresponding to a first chromosome being tested for aneuploidy, and a second set of probes comprising at least 100 different nucleic acid sequences corresponding to a reference chromosome, wherein the first chromosome being tested for aneuploidy and the reference chromosome are different;
wherein the at least one target-specific probe comprises a plurality of chromosome-specific probes;
wherein the at least one probe-specific oligonucleotide primer comprises a plurality of chromosome-specific oligonucleotide primers, wherein the plurality of chromosome-specific oligonucleotide primers comprises at least one chromosome-specific oligonucleotide primer specific for single-stranded circles derived from the first chromosome being tested for aneuploidy, and at least one chromosome-specific oligonucleotide primer specific for single-stranded circles derived from the reference chromosome;
amplifying, selectively, the double-stranded primer-circle complexes to generate linear single-stranded products,
wherein the target-specific dye-labeled detector-probe is a plurality of chromosome-specific dye-labeled detector-probes, wherein the plurality of chromosome-specific detector-probes comprises at least one chromosome-specific detector-probe that is complementary to a chromosome-specific probe from the first chromosome being tested for aneuploidy, and at least one chromosome-specific detector-probe that is complementary to a chromosome-specific probe from the reference chromosome, wherein the plurality of chromosome-specific dye-labeled detector-probes specific for the first chromosome being tested for aneuploidy is labeled with a first fluorescent dye and the plurality of chromosome-specific dye-labeled detector-probes specific for the reference chromosome is labeled with a second fluorescent dye,
wherein the presence of the chromosome-specific dye-labeled detector-probe comprising the first fluorescent dye indicates the presence of fetal aneuploidy.

42. The method of claim 41, wherein the plurality of chromosome-specific probes shares a common custom sequence.

43. The method of claim 42, wherein the common custom sequence comprises a region that is complementary to the chromosome-specific oligonucleotide primer and a region that is complementary to the chromosome-specific dye-labeled detector-probe.

44. The method of claim 41, wherein the fetal aneuploidy is selected from the group consisting of trisomy 21, trisomy 18, trisomy 13, monosomy X, triple X syndrome, XYY syndrome, and XXY syndrome.

45. A composition comprising at least one set of chromosome-specific oligonucleotide primers complementary to at least two different human chromosomes, comprising: a first set of chromosome-specific oligonucleotide primers complementary to a plurality of target sequences from a first chromosome, and a second set of chromosome-specific oligonucleotide primers complementary to a plurality of target sequences from a second chromosome.

46. A composition comprising at least one set of chromosome-specific dye-labeled detector-probes for detecting at least two human chromosomes, comprising: a first set of chromosome-specific dye-labeled detector-probes complementary to a plurality of probe-specific oligonucleotide primers specific to a first chromosome, and a second set of chromosome-specific dye-labeled detector-probes complementary to a plurality of probe-specific oligonucleotide primers specific to a second chromosome.

47. A kit comprising the composition of claim 45 and the composition of claim 46.


SEQUENCE LISTING (10 sequences)

SEQ ID NO: 1 - DNA, 23 bp, Artificial Sequence
  Synthetic oligonucleotide
ttctgacctgaaggctctgcgcg

SEQ ID NO: 2 - DNA, 23 bp, Artificial Sequence
  Synthetic oligonucleotide
ttctgacctgaaggctctgcgcg

SEQ ID NO: 3 - DNA, 19 bp, Artificial Sequence
  Synthetic oligonucleotide
agatttggacctgcgagcg

SEQ ID NO: 4 - DNA, 20 bp, Artificial Sequence
  Synthetic oligonucleotide
gagcggctgtctccacaagt

SEQ ID NO: 5 - DNA, 26 bp, Artificial Sequence
  Synthetic oligonucleotide
tcgaccgaccaccctagcgacccgta

SEQ ID NO: 6 - DNA, 26 bp, Artificial Sequence
  Synthetic oligonucleotide
tcgaccgacccttctgagctcctgcg

SEQ ID NO: 7 - DNA, 18 bp, Artificial Sequence
  Synthetic oligonucleotide
gcccgacttagcgtacca

SEQ ID NO: 8 - DNA, 24 bp, Artificial Sequence
  Synthetic oligonucleotide
ggtcggtcgatggtacgctaagtc

SEQ ID NO: 9 - DNA, 18 bp, Artificial Sequence
  Synthetic oligonucleotide
ccaccctagcgacccgta

SEQ ID NO: 10 - DNA, 18 bp, Artificial Sequence
  Synthetic oligonucleotide
cccttctgagctcctgcg